(19) United States
(12) Patent Application Publication
(10) Pub. No.: US 2023/0346873 A1
(43) Pub. Date: Nov. 2, 2023

(54) Enhanced Mediterranean Diet-Based Nutritional Compositions And Methods

(71) Applicant: DailyColors Health Inc., Sebastopol, CA (US)

(72) Inventor: Hartley Pond, Sebastopol, CA (US)

(21) Appl. No.: 18/350,891

(22) Filed: Jul. 12, 2023

Related U.S. Application Data
(63) Continuation-in-part of application No. 17/567,751, filed on Jan. 3, 2022, now Pat. No. 11,752,188.
Application No. 17/567,751 is a division of application No. 17/508,543, filed on Oct. 22, 2021, now Pat. No. 11,246,903.
Application No. 17/508,543 is a division of application No. 17/360,261, filed on Jun. 28, 2021, now Pat. No. 11,202,816.
Application No. 17/360,261 is a division of application No. 17/167,998, filed on Feb. 4, 2021, now Pat. No. 11,065,295.
(60) Provisional application No. 63/388,340, filed on Jul. 12, 2022.
(60) Provisional application No. 63/086,207, filed on Oct. 1, 2020.
(60) Provisional application No. 63/010,183, filed on Apr. 15, 2020.
(60) Provisional application No. 62/970,615, filed on Feb. 5, 2020.

(51) Int. Cl. A61K 36/9068; A23L 33/105; A23L 33/00; A61K 9/00; A61K 9/14; A61K 45/06; A61K 36/23; A61K 36/45; A61K 36/752; A61K 36/81; A61K 36/21

(52) U.S. Cl. CPC A61K 36/9068; A23L 33/105; A23L 33/30; A61K 9/0053; A61K 9/14; A61K 45/06; A61K 36/23; A61K 36/45; A61K 36/752; A61K 36/81; A61K 36/21

(57) ABSTRACT

Compositions and methods are presented in which specific compounds or extracts having known or suspected activity are combined with a plurality of chemically distinct polyphenols to achieve significantly improved biological activity. In general, it is contemplated that the enhanced activity is due to the pleiotropic effects of the compounds or extracts together with the distinct polyphenols where these compounds act synergistically on one, and more typically more than one metabolic and/or signaling pathway.

This application claims priority to US Provisional Patent Application with the Ser. No. 63/388,340, which was filed Jul. 12, 2022, and is a continuation-in-part application of allowed US patent Application with the Ser. No. 17/567,751, which was filed Jan. 3, 2022, which claims priority to U.S. Pat. No. 11,246,903, which was filed Oct. 22, 2021, which claims priority to U.S. Pat. No. 11,202,816, filed Jun. 28, 2021, which claims priority to U.S. Pat. No. 11,065,295, filed Feb. 4, 2021, which claims priority to US Provisional Patent Applications with the Ser. No. 62/970,615, filed Feb. 5, 2020, Ser. No. 63/010,183, filed Apr. 15, 2020, and Ser. No. 63/086,207, filed Oct. 1, 2020, each of which is incorporated by reference herein.

### FIELD OF THE INVENTION

The field of the invention is various compositions and methods of use for nutritional supplements, especially as it relates to specific compounds or extracts with known or suspected activity where these compounds or extracts are in synergistic combination with polyphenols and polyphenol mixtures commonly associated with a diet rich in fruits and vegetables.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

There is a considerable variety of vitamins and other isolated nutritional compounds, and alleged benefits of such compounds include, among numerous other effects, immune support, anti-inflammatory effects, anti-ageing effects, cardiac support, and digestive support. Unfortunately, there is only a rather small body of evidence that substantiates some aspects of these alleged benefits when these vitamins and other isolated nutritional compounds are ingested. Similarly, where the nutritional supplement is an extract or powdered form of a plant part, various benefits are advertised, but actual benefits are often poorly or even not at all proven. Moreover, isolated nutritional compounds as well as individual plant extracts and concentrates are generally not reflective of a healthy diet.

Still further, many of the known compounds or extracts target individual mechanisms of action or are co-factors in enzymes and as such tend to affect single points in a significantly larger network of metabolic processes. Consequently, the modulating biological activity of many of these compounds or extracts can be compensated for in a cell by way of salvage pathways, up- or down-regulation of cellular components affected by the modulated biological activity, etc.

In other known supplements, certain polyphenols are used to provide specific effects. For example, Vital Reds (by Gundry MD) is a commercially available concentrated polyphenol powder blend from a number of red colored plant materials. Here, the benefits of that composition is advertised to increase natural energy and to improve digestion. Similarly, Oxxynea by Fytexia, a commercially available mixture of grape, olive, pomegranate, green tea, grapefruit, bilberry, and orange extracts is offered as an antioxidant formulation, and the benefits of this composition were advertised to protect cells from oxidative stress and to maintain redox homeostasis.

More recently, a nutritional composition based on a synergistic combination of a variety of polyphenolic components typical of the Mediterranean diet was shown to have significant inhibitory effects on various targets in multiple and distinct pathways associated with the benefits of the Mediterranean diet (see e.g., U.S. Ser. No. 11/065,295, U.S. Ser. No. 11/202,816, U.S. Ser. No. 11/246,903, all incorporated by reference herein). While such nutritional composition demonstrated remarkable in vitro and in vivo effects, the biological effects were reflective of the various benefits of the Mediterranean diet but did not significantly extend beyond these benefits.

Thus, even though various nutritional supplements are known in the art, all or almost all of them suffer from various disadvantages. Consequently, there is a need to provide improved compositions and methods for nutritional supplements, and especially compositions in which the biological activity of specific compounds or extracts is enhanced by combination with polyphenols and polyphenol mixtures, and/or where the benefits of polyphenols mixtures are extended beyond activities otherwise associated with polyphenols.

### SUMMARY OF THE INVENTION

The inventor has discovered combinations of specific compounds or extracts having known or suspected activity with polyphenols and polyphenol mixtures enhance (typically synergistically) the known or suspected activity of the compounds or extracts. Similarly, the inventor has discovered that the activities of polyphenols and polyphenol mixtures could be extended beyond otherwise known activities using specific compounds or extracts. While not wishing to be bound by any theory or hypothesis, the inventor contemplates that the enhancement of the activity is due to a co-modulation of metabolic and/or signaling pathways that amplifies beneficial effects of the ingredients and/or that reduces compensatory effects in cells otherwise encountered.

In one aspect of the inventive subject matter, an enhanced nutritional composition that includes (1) a nutritionally acceptable compound or extract having known or suspected activity, and (2) a plurality of chemically distinct polyphenol-containing plant materials or chemically distinct polyphenols for plant materials. Most typically, the nutritional composition is formulated for oral administration, and the plant materials or chemically distinct polyphenols are present in the nutritional composition in an amount effective in increase the activity as compared to the compound or extract alone.

In some embodiments, the plant materials have a red color, a green color, an orange-yellow color, and a purple-blue color. For example, the red colored plant materials may comprise an apple extract, a pomegranate extract, a tomato powder, and/or a beet root powder; the green colored plant materials may comprise an olive extract, a rosemary extract, a green coffee bean extract, and/or a kale powder; the orange-yellow colored plant materials may comprise an onion extract, a ginger extract, a grapefruit extract, and/or a carrot powder; and the purple-blue colored plant materials may comprise a grape extract, a blueberry extract, a currant powder, and/or an elderberry powder.

In further embodiments, the nutritionally acceptable compound or extract may be a vitamin, an isolated or purified compound, an element in ionic form, a complex organic molecule of biological structure, a plant part or plant extract, an enzyme, and a probiotic.

Among other suitable choices, contemplated vitamin include vitamin A, vitamin B1, vitamin B3 (niacin) or derivative or metabolite thereof, vitamin B6, vitamin B7 (biotin), vitamin B9 (folate), vitamin B12, vitamin C, vitamin D3, vitamin E, CoQ10, and vitamin K2.

Contemplated isolated or purified compound include 5-gamma-aminobutyric acid (GABA), alpha lipoic acid (ALA), apoaequorin, a betalain, caffeine, carnitine, a ceramide, chondroitin, a conjugated linoleic acid (CLA), creatine, curcumin, cysteine, a green tea catechin (EGCG, EC, EGC, GCG, GC), cytidine 5-phosphocholine, dehydroepiandrosterone (DHEA), eicosapentaenoic Acid (EPA), a medium-chain fatty acid (MCFA), a short chain fatty acid (SCFA), an omega-3 fatty acid, a monounsaturated fatty acid (MUFA, and especially oleic acid), a polyunsaturated fatty acid (PUFA), a fructoborate, Glucosamine, hyaluronic acid, hydroxyproline, hydroxytryptophan (5-HTP), a soy isoflavone, inositol hexaphosphate, a ketone, a lignan, lecithin, lycopene, lysine, mannose, melatonin, methylsulfonylmethane (MSM), N-acetyltrosine, theanine, a phytosterol, N-acetyl-carnitine, N-acetylcysteine, N-acetyltyrosine, phosphatidylserine, a phytoestrogen, a polycosanol, quercetin, resveratrol, ribose, S-adenosylmethionine (SAM), Tryptophane, tyrosine, uridine-5-monophosphate, ursolic acid, and yohimbine.

Contemplated elements may be complexed, covalently bound, or may be in ionic form and include boron, calcium, copper, iron, molybdenum, magnesium, manganese, selenium, silicon, strontium, and zinc.

Contemplated complex organic molecules or biological structure include collagen (esp. type II), colostrum, a demineralized bone matrix, eggshell membrane, a fiber, a beta glucan, a mushroom polysaccharide, a plant or whey protein, a prebiotic, and a keratin.

Contemplated plants, plant parts, and/or extracts thereof include those where the plant is selected from the group consisting of Aloe vera, Ashwagandha spec., Bacopa monierii, Black cohosh, Boswellia spec., Chamomile, Chasteberry, Cissus quadrangularis, Coffee cherry, Cranberry, Dong Quai, Echinacea, Elderberry, Fenugreek, Garlic, Ginger, Ginkgo biloba, Ginseng, Grapefruit, Hawthorn, Horny goat, Horsetail grass, Hops, Kava, Licorice, Maca, Milk Thistle, Nettle, St. John's wort, Tongkat Ali, Passion flower, Pumpkin, Pygeum, Red yeast rice, Rhodiola, Saw palmetto, Tribulus, Turmeric, Valerian, Wild yam, and Yohimbe.

Contemplated enzymes include an amylase, bromelain, a galactosidase, papain, a lactase, a lipase, and a xylanase.

Contemplated probiotics include Lactobacillus acidophilus, Lactobacillus casei, Lactobacillus plantarum, Lactobacillus brevis, Lactobacillus gasseri, Lactobacillus rhamnosus, Bifidobacterium lactis, Bifidobacterium breve, and Bifidobacterium longum.

In further aspects of the inventive subject matter, the known or suspected activity may be support of bone and joint health, support of hair and skin health, support of cellular energy, reduction in inflammation, support of immunity, reduction in stress, support of sleep health, support of heart health, reduction of weight and/or appetite, support of men's health, support of woman's health, support of brain and/or mind health, support of mood, support of digestive health, and/or support of strength and/or fitness.

For example, where the known or suspected activity is support of bone and joint health, the nutritionally acceptable compound or extract may be selected from the group consisting of vitamin D3, vitamin K2, vitamin B3, vitamin A, a green tea catechin, chondroitin, glucosamine, calcium fructoborate, methylsulfonylmethane (MSM), Collagen type II, a hyaluronic acid, L-lysine, an eggshell membrane, calcium, magnesium, zinc, copper, strontium, an omega-3 fatty acid, a turmeric portion or extract thereof, curcumin, a Boswellia portion or extract thereof, and a demineralized bone matrix.

In another example, where the known or suspected activity is support of hair and skin health, the nutritionally acceptable compound or extract may be selected from the group consisting of an alpha lipoic acid, methylsulfonylmethane (MSM), a collagen, keratin, L-cysteine, an Aloe vera portion or extract thereof, a turmeric portion or extract thereof, a ginger portion or extract thereof, an omega-3 fatty acid, a monounsaturated fatty acid (MUFA, and especially oleic acid), a polyunsaturated fatty acid (PUFA), CoQ10, biotin, magnesium, selenium, zinc, copper, iron, silicon, vitamin B3, vitamin B6, vitamin D, vitamin E, biotin, a hyaluronic acid, hydroxyproline, a ceramide, and a Horsetail grass portion or extract thereof.

In a further example, where the known or suspected activity is support of cellular energy, the nutritionally acceptable compound or extract may be selected from the group consisting of CoQ10, a medium chain fatty acid (MCT), a ribose, niacin, nicotinamide, nicotinamide riboside, and NAD.

In yet another example, where the known or suspected activity is reduction in inflammation, the nutritionally acceptable compound or extract may be selected from the group consisting of an omega-3 fatty acid, methylsulfonylmethane (MSM), melatonin, a green tea catechin, ursolic acid, resveratrol, and a lignan.

In still another example, where the known or suspected activity is support of immunity, the nutritionally acceptable compound or extract may be selected from the group consisting of an omega-3 fatty acid, Zn+, N-acetylcysteine, a mushroom polysaccharide, an Elderberry portion or extract thereof, Vitamin C, an Echinacea portion or extract thereof, a Garlic portion or extract thereof, a probiotic, colostrum, a beta glucan, a Grapefruit portion or extract thereof, Oscillococcinum, inositol hexaphosphate, and a lignan.

In another example, where the known or suspected activity is reduction in stress, the nutritionally acceptable compound or extract may be selected from the group consisting of S-adenosylmethionine (SAM), an Ashwagandha portion or extract thereof, 5-hydroxytryptophane (5-HTP), theanine, gamma-aminobutyric acid (GABA), a Kava portion or extract thereof, a Valerian portion or extract thereof, Mg2+, vitamin B6, vitamin B12, N-acetyltrosine, and a St. John's wort portion or extract thereof.

In a further example, where the known or suspected activity is support of sleep health, the nutritionally acceptable compound or extract may be selected from the group consisting of an omega-3 fatty acid, melatonin, a Hops portion or extract thereof, a Chamomile portion or extract thereof, a Valerian portion or extract thereof, a Passion flower portion or extract thereof, and a St. John's wort portion or extract thereof.

In still another example, where the known or suspected activity is support of heart health, the nutritionally acceptable compound or extract may be selected from the group consisting of CoQ10, an omega-3 fatty acid, methylsulfonylmethane (MSM), red yeast rice, a phytosterol, a polycosanol, Niacin, a Garlic portion or extract thereof, and a Hawthorn portion or extract thereof.

In yet a further example, where the known or suspected activity is reduction of weight and/or appetite, the nutritionally acceptable compound or extract may be selected from the group consisting of an alpha lipoic acid, a medium chain fatty acid (MCFA), a short chain fatty acid (SCFA), a conjugated linoleic acid (CLA), a ketone, caffeine, theanine, carnitine, a green tea catechin, ursolic acid, resveratrol, quercetin, and a Yohimbe portion or extract thereof.

In an additional example, where the known or suspected activity is support of men's health, the nutritionally acceptable compound or extract may be selected from the group consisting of a Fenugreek portion or extract thereof, a Saw palmetto portion or extract thereof, a Pumpkin portion or extract thereof, a Nettle portion or extract thereof, a Ginger portion or extract thereof, a green tea catechin, lycopene, dehydroepiandrosterone (DHEA), a phytosterol, a Turmeric portion or extract thereof, a Pygeum portion or extract thereof, calcium, zinc, selenium, copper, molybdenum, manganese, boron, Yohimbine, a Ginkgo portion or extract thereof, a Ginseng portion or extract thereof, a Tribulus portion or extract thereof, a Horny goat portion or extract thereof, a Maca portion or extract thereof, a Tongkat Ali portion or extract thereof, vitamin E, vitamin B6, and vitamin B12.

On the other hand, where the known or suspected activity is support of woman's health, the nutritionally acceptable compound or extract may be selected from the group consisting of an omega-3 fatty acid, vitamin B1, vitamin B3, vitamin B6, vitamin B7 (biotin), vitamin B9 (folate), vitamin B12, vitamin C, vitamin D3, CoQ10, a probiotic, a Cranberry portion or extract thereof, mannose, a Black cohosh portion or extract thereof, a soy isoflavone, a phytoestrogen, a Wild yam portion or extract thereof, a Chasteberry portion or extract thereof, a Cissus quadrangularis portion or extract thereof, a Licorice portion or extract thereof, a Dong Quai portion or extract thereof, and Maca portion or extract thereof.

In a still further example, wherein the known or suspected activity is support of brain and/or mind health, the nutritionally acceptable compound or extract may be selected from the group consisting of an omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a Coffee fruit portion or extract thereof, a Ginkgo biloba portion or extract thereof, an alpha lipoic acid, apoaequorin, and uridine-5-monophosphate.

Moreover, where the known or suspected activity is support of mood, the nutritionally acceptable compound or extract may be selected from the group consisting of dehydroepiandrosterone (DHEA), S-adenosylmethionine (SAM), eicosapentaenoic acid (EPA), a Bacopa monierii portion or extract thereof, caffeine, 5-hydroxytryptophane (5-HTP), tryptophan, and a St. John's wort portion or extract thereof.

Additionally, where the known or suspected activity is support of digestive health, the nutritionally acceptable compound or extract may be selected from the group consisting of an alpha lipoic acid, a prebiotic, a probiotic, bromelain, papain, a Ginger portion or extract thereof, an amylase, a lipase, a lactase, a galactosidase, a xylanase, a short chain fatty acid (SCFA), N-acetylcysteine, a Milk Thistle portion or extract thereof, a soluble fiber, and an insoluble fiber.

In yet another example, where the known or suspected activity is support of strength and/or fitness, the nutritionally acceptable compound or extract may be selected from the group consisting of CoQ10, a ketone, creatine, a betalains, a plant protein, and a whey protein.

In some embodiments, the composition is formulated in single dosage units for oral administration, for example, in a dosage unit that contains between 50 and 1,000 mg of the composition. Most typically, but not necessarily, the dosage unit is formulated as a capsule, a gummy, or a powder.

Consequently, an exemplary an enhanced nutritional composition may include (1) a nutritionally acceptable compound or extract having a known or suspected activity selected from the group consisting of support of bone and joint health, support of hair and skin health, support of cellular energy, reduction in inflammation, support of immunity, reduction in stress, support of sleep health, support of heart health, reduction of weight and/or appetite, support of men's health, support of woman's health, support of brain and/or mind health, support of mood, support of digestive health, and support of strength and/or fitness; and (2) a plurality of chemically distinct polyphenol-containing plant materials or chemically distinct polyphenols from plant materials, wherein the plant materials have a red color, a green color, an orange-yellow color, and a purple-blue color.

In some embodiments, the red colored plant materials may comprise an apple extract, a pomegranate extract, a tomato powder, and/or a beet root powder; the green colored plant materials may comprise an olive extract, a rosemary extract, a green coffee bean extract, and/or a kale powder; the orange-yellow colored plant materials may comprise an onion extract, a ginger extract, a grapefruit extract, and/or a carrot powder; and the purple-blue colored plant materials may comprise a grape extract, a blueberry extract, a currant powder, and/or an elderberry powder.

Most typically, the enhanced nutritional composition will be formulated in single dosage units for oral administration (e.g., the dosage unit contains between 50 and 1,000 mg of the composition), and the dosage unit is formulated as a capsule, a gummy, or a powder.

For example, the dosage unit may contain vitamin D3, vitamin K2, vitamin B3, vitamin A, a green tea catechin, chondroitin, glucosamine, calcium fructoborate, methylsulfonylmethane (MSM), Collagen type II, a hyaluronic acid, L-lysine, an eggshell membrane, calcium, magnesium, zinc, copper, strontium, an omega-3 fatty acid, a turmeric portion or extract thereof, curcumin, a Boswellia portion or extract thereof, and/or a demineralized bone matrix as the compound or extract and, upon oral administration to a subject, supports bone and joint health in the subject.

In another example, the dosage unit may contain alpha lipoic acid, methylsulfonylmethane (MSM), a collagen, keratin, L-cysteine, an Aloe vera portion or extract thereof, a turmeric portion or extract thereof, a ginger portion or extract thereof, an omega-3 fatty acid, a monounsaturated fatty acid (MUFA, and especially oleic acid), a polyunsaturated fatty acid (PUFA), CoQ10, biotin, magnesium, selenium, zinc, copper, iron, silicon, vitamin B3, vitamin B6, vitamin D, vitamin E, biotin, a hyaluronic acid, hydroxyproline, a ceramide, and/or a Horsetail grass portion or extract thereof as the compound or extract and, upon oral administration to a subject, supports hair and skin health in the subject.

In a further example, the dosage unit may contain CoQ10, a medium chain fatty acid (MCT), a ribose, niacin, nicotinamide, nicotinamide riboside, and/or NAD as the compound or extract and, upon oral administration to a subject, supports cellular energy in the subject.

In still a further example, the dosage unit may contain an omega-3 fatty acid, methylsulfonylmethane (MSM), melatonin, a green tea catechin, ursolic acid, resveratrol, and/or a lignan as the compound or extract and, upon oral administration to a subject, reduces inflammation in the subject.

In yet a further example, the dosage unit may contain an omega-3 fatty acid, Zn+, N-acetylcysteine, a mushroom polysaccharide, an Elderberry portion or extract thereof, Vitamin C, an Echinacea portion or extract thereof, a Garlic portion or extract thereof, a probiotic, colostrum, a beta glucan, a Grapefruit portion or extract thereof, Oscillococcinum, inositol hexaphosphate, and/or a lignan as the compound or extract and, upon oral administration to a subject, supports immunity in the subject.

Moreover, the dosage unit may contain S-adenosylmethionine (SAM), an Ashwagandha portion or extract thereof, 5-hydroxytryptophane (5-HTP), theanine, gamma-aminobutyric acid (GABA), a Kava portion or extract thereof, a Valerian portion or extract thereof, Mg2+, vitamin B6, vitamin B12, N-acetyltrosine, and/or a St. John's wort portion or extract thereof as the compound or extract and, upon oral administration to a subject, reduces stress in the subject.

Additionally, the dosage unit may contain an omega-3 fatty acid, melatonin, a Hops portion or extract thereof, a Chamomile portion or extract thereof, a Valerian portion or extract thereof, a Passion flower portion or extract thereof, and/or a St. John's wort portion or extract thereof as the compound or extract and, upon oral administration to a subject, supports sleep health in the subject.

In yet another example, the dosage unit may contain CoQ10, an omega-3 fatty acid, methylsulfonylmethane (MSM), red yeast rice, a phytosterol, a polycosanol, Niacin, a Garlic portion or extract thereof, and/or a Hawthorn portion or extract thereof as the compound or extract and, upon oral administration to a subject, supports heart health in the subject.

In a still further example, the dosage unit may contain an alpha lipoic acid, a medium chain fatty acid (MCFA), a short chain fatty acid (SCFA), a conjugated linoleic acid (CLA), a ketone, caffeine, theanine, carnitine, a green tea catechin, ursolic acid, resveratrol, quercetin, and/or a Yohimbe portion or extract thereof as the compound or extract and, upon oral administration to a subject, reduces weight and/or appetite in the subject.

In an additional example, the dosage unit may contain Fenugreek portion or extract thereof, a Saw palmetto portion or extract thereof, a Pumpkin portion or extract thereof, a Nettle portion or extract thereof, a Ginger portion or extract thereof, a green tea catechin, lycopene, dehydroepiandrosterone (DHEA), a phytosterol, a Turmeric portion or extract thereof, a Pygeum portion or extract thereof, calcium, zinc, selenium, copper, molybdenum, manganese, boron, Yohimbine, a Ginkgo portion or extract thereof, a Ginseng portion or extract thereof, a Tribulus portion or extract thereof, a Horny goat portion or extract thereof, a Maca portion or extract thereof, a Tongkat Ali portion or extract thereof, vitamin E, vitamin B6, and/or vitamin B12 as the compound or extract and, upon oral administration to a subject, supports men's health in the subject.

Likewise, the dosage unit may contain an omega-3 fatty acid, vitamin B1, vitamin B3, vitamin B6, vitamin B7 (biotin), vitamin B9 (folate), vitamin B12, vitamin C, vitamin D3, CoQ10, a probiotic, a Cranberry portion or extract thereof, mannose, a Black cohosh portion or extract thereof, a soy isoflavone, a phytoestrogen, a Wild yam portion or extract thereof, a Chasteberry portion or extract thereof, a Cissus quadrangularis portion or extract thereof, a Licorice portion or extract thereof, a Dong Quai portion or extract thereof, and/or Maca portion or extract thereof as the compound or extract and, upon oral administration to a subject, improves women's health in the subject.

Additionally, a dosage unit may contain an omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a Coffee fruit portion or extract thereof, a Ginkgo biloba portion or extract thereof, an alpha lipoic acid, apoaequorin, and/or uridine-5-monophosphateas the compound or extract and, upon oral administration to a subject, supports brain and/or mind health in the subject.

Moreover, a dosage unit may contain dehydroepiandrosterone (DHEA), S-adenosylmethionine (SAM), eicosapentaenoic acid (EPA), a Bacopa monierii portion or extract thereof, caffeine, 5-hydroxytryptophane (5-HTP), tryptophan, and/or a St. John's wort portion or extract thereof as the compound or extract and, upon oral administration to a subject, supports mood in the subject.

In yet another example, the dosage unit may contain an alpha lipoic acid, a prebiotic, a probiotic, bromelain, papain, a Ginger portion or extract thereof, an amylase, a lipase, a lactase, a galactosidase, a xylanase, a short chain fatty acid (SCFA), N-acetylcysteine, a Milk Thistle portion or extract thereof, a soluble fiber, and/or an insoluble fiber as the compound or extract and, upon oral administration to a subject, improves digestive health in the subject.

In a still further example, the dosage unit may contain CoQ10, a ketone, creatine, a betalains, a plant protein, and a whey protein as the compound or extract and, upon oral administration to a subject, improves strength or fitness in the subject.

Viewed form a different perspective, the inventor also contemplates a method of enhancing a biological activity of a nutritionally acceptable compound or extract that includes a step of combining the nutritionally acceptable compound or extract with a plurality of chemically distinct polyphenol-containing plant materials or chemically distinct polyphenols from plant materials to thereby form an enhanced composition. Preferably, the plurality of chemically distinct polyphenol-containing plant materials or chemically distinct polyphenols are present in the enhanced composition in an amount effective to enhance the biological activity of the nutritionally acceptable compound or extract as compared to the biological activity of the nutritionally acceptable compound or extract alone; and the enhanced biological activity is observed in a subject upon oral administration to the subject.

For example, where the biological activity is support of bone and joint health, the nutritionally acceptable compound or extract may be selected from the group consisting of vitamin D3, vitamin K2, vitamin B3, vitamin A, a green tea catechin, chondroitin, glucosamine, calcium fructoborate, methylsulfonylmethane (MSM), Collagen type II, a hyaluronic acid, L-lysine, an eggshell membrane, calcium, magnesium, zinc, copper, strontium, an omega-3 fatty acid, a turmeric portion or extract thereof, curcumin, a Boswellia portion or extract thereof, and a demineralized bone matrix.

In another example, where the biological activity is support of hair and skin health, the nutritionally acceptable compound or extract may be selected from the group consisting of an alpha lipoic acid, methylsulfonylmethane (MSM), a collagen, keratin, L-cysteine, an Aloe vera portion or extract thereof, a turmeric portion or extract thereof, a ginger portion or extract thereof, an omega-3 fatty acid, a monounsaturated fatty acid (MUFA, and especially oleic acid), a polyunsaturated fatty acid (PUFA), CoQ10, biotin, magnesium, selenium, zinc, copper, iron, silicon, vitamin B3, vitamin B6, vitamin D, vitamin E, biotin, a hyaluronic acid, hydroxyproline, a ceramide, and a Horsetail grass portion or extract thereof.

In a further example, where the biological activity is support of cellular energy, the nutritionally acceptable compound or extract may be selected from the group consisting of CoQ10, a medium chain fatty acid (MCT), a ribose, niacin, nicotinamide, nicotinamide riboside, and NAD.

In still a further example, where the biological activity is reduction in inflammation, the nutritionally acceptable compound or extract may be selected from the group consisting of an omega-3 fatty acid, methylsulfonylmethane (MSM), melatonin, a green tea catechin, ursolic acid, resveratrol, and a lignan.

In another example, where the biological activity is support of immunity, the nutritionally acceptable compound or extract may be selected from the group consisting of an omega-3 fatty acid, Zn+, N-acetylcysteine, a mushroom polysaccharide, an Elderberry portion or extract thereof, Vitamin C, an Echinacea portion or extract thereof, a Garlic portion or extract thereof, a probiotic, colostrum, a beta glucan, a Grapefruit portion or extract thereof, Oscillococcinum, inositol hexaphosphate, and a lignan.

In yet another example, where the biological activity is reduction in stress, the nutritionally acceptable compound or extract may be selected from the group consisting of S-adenosylmethionine (SAM), an Ashwagandha portion or extract thereof, 5-hydroxytryptophane (5-HTP), theanine, gamma-aminobutyric acid (GABA), a Kava portion or extract thereof, a Valerian portion or extract thereof, Mg2+, vitamin B6, vitamin B12, N-acetyltrosine, and a St. John's wort portion or extract thereof.

In still another example, where the biological activity is support of sleep health, the nutritionally acceptable compound or extract may be selected from the group consisting of an omega-3 fatty acid, melatonin, a Hops portion or extract thereof, a Chamomile portion or extract thereof, a Valerian portion or extract thereof, a Passion flower portion or extract thereof, and a St. John's wort portion or extract thereof.

Additionally, where the biological activity is support of heart health, the nutritionally acceptable compound or extract may be selected from the group consisting of CoQ10, an omega-3 fatty acid, methylsulfonylmethane (MSM), red yeast rice, a phytosterol, a polycosanol, Niacin, a Garlic portion or extract thereof, and a Hawthorn portion or extract thereof.

Moreover, where the biological activity is reduction of weight and/or appetite, the nutritionally acceptable compound or extract may be selected from the group consisting of an alpha lipoic acid, a medium chain fatty acid (MCFA), a short chain fatty acid (SCFA), a conjugated linoleic acid (CLA), a ketone, caffeine, theanine, carnitine, a green tea catechin, ursolic acid, resveratrol, quercetin, and a Yohimbe portion or extract thereof.

In still another example, where the biological activity is support of men's health, the nutritionally acceptable compound or extract may be selected from the group consisting of a Fenugreek portion or extract thereof, a Saw palmetto portion or extract thereof, a Pumpkin portion or extract thereof, a Nettle portion or extract thereof, a Ginger portion or extract thereof, a green tea catechin, lycopene, dehydroepiandrosterone (DHEA), a phytosterol, a Turmeric portion or extract thereof, a Pygeum portion or extract thereof, calcium, zinc, selenium, copper, molybdenum, manganese, boron, Yohimbine, a Ginkgo portion or extract thereof, a Ginseng portion or extract thereof, a Tribulus portion or extract thereof, a Horny goat portion or extract thereof, a Maca portion or extract thereof, a Tongkat Ali portion or extract thereof, vitamin E, vitamin B6, and vitamin B12.

Similarly, where the biological activity is support of woman's health, the nutritionally acceptable compound or extract is selected from the group consisting of an omega-3 fatty acid, vitamin B1, vitamin B3, vitamin B6, vitamin B7 (biotin), vitamin B9 (folate), vitamin B12, vitamin C, vitamin D3, CoQ10, a probiotic, a Cranberry portion or extract thereof, mannose, a Black cohosh portion or extract thereof, a soy isoflavone, a phytoestrogen, a Wild yam portion or extract thereof, a Chasteberry portion or extract thereof, a Cissus quadrangularis portion or extract thereof, a Licorice portion or extract thereof, a Dong Quai portion or extract thereof, and Maca portion or extract thereof.

In yet another example, where the biological activity is support of brain and/or mind health, the nutritionally acceptable compound or extract may be selected from the group consisting of an omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a Coffee fruit portion or extract thereof, a Ginkgo biloba portion or extract thereof, an alpha lipoic acid, apoaequorin, and uridine-5-monophosphate.

In a further example, where the biological activity is support of mood, the nutritionally acceptable compound or extract may be selected from the group consisting of dehydroepiandrosterone (DHEA), S-adenosylmethionine (SAM), eicosapentaenoic acid (EPA), a Bacopa monierii portion or extract thereof, caffeine, 5-hydroxytryptophane (5-HTP), tryptophan, and a St. John's wort portion or extract thereof.

In still a further example, where the biological activity is support of digestive health, and wherein the nutritionally acceptable compound or extract is selected from the group consisting of an alpha lipoic acid, a prebiotic, a probiotic, bromelain, papain, a Ginger portion or extract thereof, an amylase, a lipase, a lactase, a galactosidase, a xylanase, a short chain fatty acid (SCFA), N-acetylcysteine, a Milk Thistle portion or extract thereof, a soluble fiber, and an insoluble fiber.

In yet another example, where the biological activity is support of strength and/or fitness, the nutritionally acceptable compound or extract is selected from the group consisting of CoQ10, a ketone, creatine, a betalains, a plant protein, and a whey protein.

In some embodiments, the plant materials have a red color, a green color, an orange-yellow color, and a purple-blue color. For example, the red colored plant materials may comprise an apple extract, a pomegranate extract, a tomato powder, and/or a beet root powder; the green colored plant materials may comprise an olive extract, a rosemary extract, a green coffee bean extract, and/or a kale powder; the orange-yellow colored plant materials may comprise an onion extract, a ginger extract, a grapefruit extract, and/or a carrot powder; and the purple-blue colored plant materials may comprise a grape extract, a blueberry extract, a currant powder, and/or an elderberry powder. Preferably, but not necessarily, the enhanced composition is formulated for oral administration.

In a still further aspect of the inventive subject matter, the inventor also contemplates a method of enhancing a biological activity of a plurality of chemically distinct polyphenol-containing plant materials or chemically distinct polyphenols from plant materials. Such method will typically include a step of combining the plurality of chemically distinct polyphenol-containing plant materials or chemically distinct polyphenols with a nutritionally acceptable compound or extract to thereby form an enhanced composition. Preferably, the nutritionally acceptable compound or extract is present in the enhanced composition in an amount effective to enhance the biological activity of the plurality of chemically distinct polyphenol-containing plant materials or chemically distinct polyphenols as compared to the plurality of chemically distinct polyphenol-containing plant materials or chemically distinct polyphenols alone. Most typically, the enhanced biological activity is observed in a subject upon oral administration to the subject.

For example, the biological activity may be immune support, metabolic support, support of longevity, support of central nervous system (CNS) function, reduction of an inflammatory response, reduction of effects of cardiovascular disease, and/or reduction of the rate of amyloid beta plaque formation, and wherein the plant materials have a red color, a green color, an orange-yellow color, and a purple-blue color. Therefore, and among other options, the biological activity may be an inhibition of ARG-1, ARG-2, SIRT-1, Keap/Nrf-2, BACE-1, Cathepsin S, CDK5, IDO1, IDO2, NAMPT, PCSK9, CD47, CD38, CD39, CD73, JAK1, JAK2, and/or JAK3.

Most typically, but not necessarily, the plant materials will have a red color, a green color, an orange-yellow color, and a purple-blue color. For example, the red colored plant materials may comprise an apple extract, a pomegranate extract, a tomato powder, and a beet root powder; the green colored plant materials may comprise an olive extract, a rosemary extract, a green coffee bean extract, and/or a kale powder; the orange-yellow colored plant materials may comprise an onion extract, a ginger extract, a grapefruit extract, and/or a carrot powder; and the purple-blue colored plant materials may comprise a grape extract, a blueberry extract, a currant powder, and/or an elderberry powder.

In further embodiments, the nutritionally acceptable compound or extract may be a vitamin, an isolated or purified compound, an element, a complex organic molecule or biological structure, a plant part and/or extract thereof, and enzyme, and/or a probiotic.

Suitable vitamins include vitamin A, vitamin B1, vitamin B3 (niacin) or derivative or metabolite thereof (e.g., nicotinamide, nicotinamide riboside, NAD), vitamin B6, vitamin B7 (biotin), vitamin B9 (folate), vitamin B12, vitamin C, vitamin D3, vitamin E, and vitamin K2, or CoQ10.

Suitable isolated or purified compounds include 5-gamma-aminobutyric acid (GABA), alpha lipoic acid (ALA), apoaequorin, a betalain, caffeine, carnitine, a ceramide, chondroitin, a conjugated linoleic acid (CLA), creatine, curcumin, cysteine, a green tea catechin (EGCG, EC, EGC, GCG, GC), cytidine 5-phosphocholine, dehydroepiandrosterone (DHEA), eicosapentaenoic Acid (EPA), a medium-chain fatty acid (MCFA), a short chain fatty acid (SCFA), an omega-3 fatty acid, a monounsaturated fatty acid (MUFA, and especially oleic acid), a polyunsaturated fatty acid (PUFA), a fructoborate, Glucosamine, hyaluronic acid, hydroxyproline, hydroxytryptophan (5-HTP), a soy isoflavone, inositol hexaphosphate, a ketone (beta hydroxybutyrate, acetoacetate, acetone, . . . ), a lignan (sesamine, etc), lecithin, lycopene, lysine, mannose, melatonin, methyl sulfonylmethane (MSM), N-acetyltrosine, theanine, a phytosterol (e.g., beta sitosterol), N-acetyl-carnitine, N-acetylcysteine, N-acetyltyrosine, phosphatidylserine, a phytoestrogen, a polycosanol, quercetin, resveratrol, ribose, S-adenosylmethionine (SAM), Tryptophane, tyrosine, uridine-5-monophosphate, ursolic acid, and yohimbine.

Suitable elements may be in ionic or complexed form and include B, Ca, Cu, Fe, Mb, Mg, Mn, Se, Si, Sr, and Zn.

Suitable complex organic molecules or biological structure include collagen, colostrum, a demineralized bone matrix, eggshell membrane, fibers, beta glucans, mushroom polysaccharides, plant or whey protein, prebiotics (e.g., FOS, XOS, HMS) and keratin.

Suitable plants, plant parts, or extract thereof include those from Aloe vera, Ashwagandha, Bacopa monierii, Black cohosh, Boswellia, Chamomile, Chasteberry, Cissus quadrangularis, Coffee cherry, Cranberry, Dong Quai, Echinacea, Elderberry, Fenugreek, Garlic, Ginger, Ginkgo biloba, Ginseng, Grapefruit, Hawthorn, Horny goat, Horsetail grass, Hops, Kava, Licorice, Maca, Milk Thistle, Nettle, St. John's wort, Tongkat Ali, Passion flower, Pumpkin, Pygeum, Red yeast rice, Rhodiola, Saw palmetto, Tribulus, Turmeric, Valerian, Wild yam, and/or Yohimbe.

Suitable enzymes include amylase, bromelain, galactosidase, papain, lactase, lipase, and xylanase.

Suitable probiotics include Lactobacillus acidophilus, Lactobacillus casei, Lactobacillus plantarum, Lactobacillus brevis, Lactobacillus gasseri, Lactobacillus rhamnosus, Bifidobacterium lactis, Bifidobacterium breve, and Bifidobacterium longum.

Consequently, in yet another aspect of the inventive subject matter, the inventor also contemplates method of supporting health of an individual that includes a step of administering to the individual a composition as presented herein. For example, the composition may be administered to thereby provide immune support, metabolic support, support longevity, support central nervous system (CNS) function, reduce an inflammatory response, reduce effects of cardiovascular disease, and/or reduce the rate of amyloid beta plaque formation. Likewise, the composition may be administered to thereby provide support of bone and joint health, support of hair and skin health, support of cellular energy, reduction in inflammation, support of immunity, reduction in stress, support of sleep health, support of heart health, reduction of weight and/or appetite, support of men's health, support of woman's health, support of brain and/or mind health, support of mood, support of digestive health, and/or support of strength and/or fitness. Most typically, the composition will be orally administered over at least 30 days and/or at a daily dose of between 50 and 1,000 mg.

Various objects, features, aspects, and advantages of the inventive subject matter will become more apparent from the following detailed description of preferred embodiments.

### DETAILED DESCRIPTION

The inventor has discovered that combinations of specific compounds or extracts having known or suspected activity with polyphenols and polyphenol mixtures (e.g., those commonly associated with a diet rich in fruits and vegetables) exhibit enhanced biological activities in a pleiotropic and typically synergistic manner. While not wishing to be bound by any specific theory or hypothesis, the inventor contemplates that the enhanced activity may be attributed to inhibition and/or stimulation of multiple metabolic and/or signaling pathways that are functionally connected to the biological activities. Viewed form a different perspective, by combining the compounds/extracts with the polyphenol mixtures, biological activity will be enhanced in a manner otherwise not achievable by administration of the compounds/extracts or polyphenols alone.

Accordingly, the inventor contemplates various compositions for nutritional supplements and other nutritional products, compositions, and uses in medicinal food, and even use in medicine. Most preferably, such compositions will include first and second components (even more preferably in synergistic quantities) in which the first component comprises one or more specific compounds or extracts with known or suspected activity and in which the second component comprises a plurality of chemically distinct polyphenols. Viewed from a different perspective, the compositions presented herein will not only provide various benefits associated with the polyphenols and polyphenol mixtures but also (typically synergistically) enhance the known or suspected activity of specific compounds and/or extracts.

With respect to the compounds and/or extracts, it is contemplated that all nutritionally acceptable ingredients are deemed suitable for use herein, and especially preferred compounds and/or extracts include various vitamins, isolated or purified compounds, chemical elements, complex organic molecules or biological structures, plants, plant parts, and extracts thereof, enzymes, and probiotics.

Suitable vitamins will typically include all known vitamins, and it is contemplated that the vitamins will be present in the compositions presented herein in quantities consistent with common use in the art (which may or may not exceed the FDA recommended RDA for the specific vitamin). Therefore, and among other suitable choices, preferred vitamins include vitamin A, vitamin B1, vitamin B3 (niacin) or derivative or metabolite thereof (e.g., nicotinamide (also known as niacinamide), nicotinamide riboside, NAD), vitamin B6, vitamin B7 (biotin), vitamin B9 (folate), vitamin B12, vitamin C, vitamin D3, vitamin E, vitamin K2, and CoQ10.

With respect to the isolated or purified compounds it is generally preferred that such compounds will have known or suspected biological activity, and among other suitable choices, especially contemplated compounds include 5-gamma-aminobutyric acid (GABA), alpha lipoic acid (ALA), apoaequorin, one or more betalains (e.g., betaxanthins or betacyanins), caffeine, carnitine, a ceramide, chondroitin and various derivatives thereof (e.g., chondroitin sulfate), a conjugated linoleic acid (CLA), creatine, curcumin, cysteine, a green tea catechin (EGCG, EC, EGC, GCG, GC), cytidine 5-phosphocholine, dehydroepiandrosterone (DHEA), eicosapentaenoic Acid (EPA), a medium-chain fatty acid (MCFA), a short chain fatty acid (SCFA, typically between 4 and 8 carbon atoms in linear length), one or more omega-3 fatty acids (e.g., α-linolenic acid (ALA), fisetin and its metabolites, eicosapentaenoic acid (EPA) and docosahexaenoic acid (DHA)), a monounsaturated fatty acid (MUFA, and especially oleic acid and other fatty acids having between 14-20 carbon atoms, typically with a double bond in omega-3, 6, 7, or 9 position), a polyunsaturated fatty acid (PUFA, typically including 18 or 20 carbon atoms with double bond at 3, 6, or 9 position), calcium fructoborate, glucosamine, hyaluronic acid (low as well as high-molecular weight), hydroxyproline, hydroxytryptophan (5-HTP), one or more soy isoflavones (e.g., daidzein, genistein, glycitein, etc.) and their metabolites and glycosides, inositol hexaphosphate, various ketones (e.g., beta hydroxybutyrate, acetoacetate, acetone), a lignan (sesamine, sesamolin, sesaminol), various lecithins, lycopene, lysine, mannose, melatonin, methyl sulfonylmethane (MSM), N-acetyltrosine, theanine, a phytosterol (e.g., beta sitosterol), N-acetyl-carnitine, N-acetylcysteine, N-acetyltyrosine, phosphatidylserine, a phytoestrogen, a polycosanol, quercetin, resveratrol, ribose, S-adenosylmethionine (SAM), Tryptophane, tyrosine, uridine-5-monophosphate, ursolic acid, and yohimbine.

Contemplated elements include all micro- and macronutrients, and especially minerals and elements in complexed form (e.g., as chelate, complexed in a food matrix), covalently bound form (e.g., as organometallic compound), and in ionic form as an inorganic salt (e.g., zinc chloride), a mixed salt (e.g., zinc gluconate), or an oxide (e.g., Zn₂O). Therefore, and among other choices, boron, calcium, copper, iron, magnesium, molybdenum, manganese, selenium, silicon, strontium, and zinc.

With respect to complex organic molecules or biological structure selected it is contemplated that especially preferred compounds include collagen (e.g., type II), colostrum, demineralized bone matrix, eggshell membrane, various fibers (soluble and insoluble), various beta glucans, various mushroom polysaccharides (e.g., from turkey tail, lion's mane, shiitake, cordyceps, reishi, etc.), plant or whey protein, prebiotics (e.g., various fructooligosaccharides, xylooligosaccharides, and human milk oligosaccharides), and keratin. As should be readily appreciated, these molecules and structures may be further processed (e.g., partial or total hydrolysis, size fractionation, etc.)

In further contemplated embodiments, suitable compounds and/or extracts will include plants, plant parts (e.g., root, fruits, seed, leaves/stems, etc.), and extracts thereof, and it should be noted that the extracts may be crude pressed juices, aqueous or hydroalcoholic extracts, etc., which may be further processed to enrich in one or more desirable components using protocols well known in the art. For example, suitable plants for the compounds or extracts include Aloe vera, Ashwagandha, Bacopa monierii, Black cohosh, Boswellia, Chamomile, Chasteberry, Cissus quadrangularis, Coffee cherry, Cranberry, Dong Quai, Echinacea, Elderberry, Fenugreek, Garlic, Ginger, Ginkgo biloba, Ginseng, Grapefruit, Hawthorn, Horny goat, Horsetail grass, Hops, Kava, Licorice, Maca, Milk Thistle, Nettle, St. John's wort, Tongkat Ali, Passion flower, Pumpkin, Pygeum, Red yeast rice, Rhodiola, Saw palmetto, Tribulus, Turmeric, Valerian, Wild yam, and Yohimbe.

Especially contemplated enzymes for the compounds and/or extracts include various proteases, carbohydrate hydrolases, and lipases. Therefore, suitable enzymes include amylase, galactosidase, lactase, xylanase, bromelain, papain, and lipase.

With respect to probiotics suitable for use herein, it should be appreciated that all known probiotics are deemed suitable for use herein, and especially contemplated probiotics include bacterial and yeast probiotics. For example, suitable probiotics include various lactobacilli such as Lactobacillus acidophilus, Lactobacillus brevis, Lactobacillus casei, Lactobacillus plantarum, Lactobacillus brevis, Lactobacillus gasseri, Lactobacillus plantarum, Lactobacillus paraplantarum, Lactobacillus coryniformis, Lactobacillus rhamnosus, various bifidobacterial such as Bifidobacterium lactis, Bifidobacterium breve, and Bifidobacterium longum, Leuconostoc mesenteroides, Pediococcus pentosaceus, Leuconostoc citreum, Leuconostoc argentinum, Lactococcus sp., and Weissella ssp.

Consequently, and depending on the particular choice of compounds and/or extracts, it should be appreciated that the known or suspected activities associated with the compounds and extracts will vary considerably.

In a first example, where the compounds and/or extract is vitamin D3, vitamin K2, vitamin B3, vitamin A, a green tea catechin (e.g., EGCG, ECG, EC), chondroitin, glucosamine, Ca Fructoborate, MSM, collagen type II, hyaluronic acid, L-Lysine, eggshell membrane, calcium, magnesium, zinc, coper, strontium, an omega-3 fatty acid, Turmeric or a turmeric extract, curcumin, Boswellia or a Boswellia extract, and/or demineralized bone matrix, the associated activity may provide support for bone and/or joint health.

In a second example, where the compounds and/or extract is an alpha lipoic acid, MSM, a collagen, keratin, cysteine, Aloe vera or a Aloe vera extract, Turmeric or a Turmeric extract, Ginger or a Ginger extract, an omega-3 fatty acid, a MUFA, a PUFA, CoQ10, biotin, magnesium, selenium, zinc, copper, iron, silicon, vitamin B3, vitamin B6, vitamin D, vitamin E biotin, hyaluronic acid, hydroxyproline, a ceramide, and/or Horsetail grass or a Horsetail grass extract, the associated activity may provide support for skin and/or hair.

In a third example, where the compounds and/or extract is CoQ10, a medium-chain triglyceride, ribose, niacin, nicotinamide, nicotinamide riboside, and/or NAD, the associated activity may provide support for cellular energy.

In a fourth example, where the compounds and/or extract is an omega-3 fatty acid, MSM, melatonin, a green tea catechin, ursolic acid, resveratrol, and/or a lignan, the associated activity may reduce inflammation.

In a fifth example, where the compounds and/or extract is an omega-3 fatty acid, zinc, N-acetylcysteine, a mushroom polysaccharide (e.g., from turkey tail, lion's mane, shiitake, cordyceps, reishi, etc.), elderberry or an elderberry extract, vitamin C, echinacea or an echinacea extract, a garlic extract, a probiotics, colostrum, a beta glucan, a grapefruit seed extract, Oscillococcinum, Inositol hexaphosphate, and/or a lignan, the associated activity may provide support for immunity/proper immune function.

In a sixth example, where the compounds and/or extract is SAM, Ashwagandha or an Ashwagandha extract, 5-HTP, theanine, GABA, a kava extract, a valerian extract, magnesium, vitamin B6, vitamin B12, N-acetyltrosine, and/or St. John's wort, the associated activity may reduce stress.

In a seventh example, where the compounds and/or extract is an omega-3 fatty acid, melatonin, Hops or a Hops extract, Chamomile or a Chamomile extract, Valerian or a Valerian extract, Passion flower or a Passion flower extract, and/or St. John's wort or a St. John's wort extract, the associated activity may provide support for healthy sleep.

In an eighth example, where the compounds and/or extract is CoQ10, an omega-3 fatty acid, MSM, Red yeast rice, a phytosterol (e.g., beta sitosterol), a polycosanol, niacin, a garlic extract, and/or a Hawthorn extract, the associated activity may provide support for heart health.

In a ninth example, where the compounds and/or extract is alpha lipoic acid, a MCFA, a SCFA, a CLA, a ketone (e.g., BHB, acetoacetate, etc.), caffeine, theanine, carnitine, a green tea catechin, ursolic acid, fisetin, resveratrol, quercetin, and/or a Yohimbe bark extract, the associated activity may provide support for weight loss and/or appetite control.

In a tenth example, where the compounds and/or extract is a fenugreek extract, a saw palmetto extract, a pumpkin seed extract, a nettle extract, a ginger extract, a green tea catechin, lycopene, DHEA, a phytosterols, a turmeric extract, a pygeum bark extract, calcium, zinc, selenium, copper molybdenum, manganese, boron, yohimbine, a Ginkgo extract, a Ginseng extract, a Tribulus extract, a Horny goat extract, a Maca root extract, a Tongkat Ali extract, vitamin E, vitamin B6, and/or vitamin B12, the associated activity may provide support for men's health.

In an eleventh example, where the compounds and/or extract is an omega-3 fatty acid, vitamin B1, vitamin B3, vitamin B6, vitamin B12, vitamin C, vitamin D3, CoQ10, biotin, folate, a probiotics, a cranberry extract, mannose, a black cohosh extract, a soy isoflavone, a phytoestrogen, a wild yam extract, a chasteberry extract, a Cissus quadrangularis extract, a licorice root extract, a Dong Quai root extract, and/or a Maca root extract, the associated activity may provide support for women's health.

In a twelfth example, where the compounds and/or extract is an omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a coffee cherry extract, Ginkgo biloba or a Ginkgo biloba extract, alpha lipoic acid, apoaequorin, and/or Uridine-5-monophosphate, the associated activity may provide support for mind and/or brain health.

In a thirteenth example, where the compounds and/or extract is DHEA, SAM, EPA (Eicosapentaenoic Acid), Bacopa monierii or a Bacopa monierii extract, caffeine, 5-HTP, tryptophan, and/or St. John's wort or a St. John's wort extract, the associated activity may provide support for or improve mood.

In a fourteenth example, where the compounds and/or extract is alpha lipoic acid, MSM, melatonin, fisetin, a green tea catechin, resveratrol, and/or a Rhodiola extract, the associated activity may provide antioxidant effect.

In a fifteenth example, where the compounds and/or extract is alpha lipoic acid, a prebiotic (e.g., FOS, XOS, HMO, etc.), a probiotic, bromelain, papain, ginger or a ginger extract, an amylase, a lipase, a lactase, a galactosidase, a xylanase, a short chain fatty acid (SCFA), N-acetylcysteine, a milk thistle extract, and/or a dietary fiber (soluble and insoluble), the associated activity may provide digestive support.

In a sixteenth example, where the compounds and/or extract is CoQ10, a ketone, creatine, a betalain, and/or a plant and whey protein, the associated activity may increase strength or fitness.

Therefore, and viewed from a different perspective, the compounds and/or extracts presented herein may be used to maintain, support, or even improve bone and joint health, hair and skin health, cellular energy, immunity/proper immune response, heart health, support of men's health, woman's health, brain and/or mind health, mood, sleep, digestive health, strength and/or fitness and/or may reduce inflammation, (oxidative) stress, and/or weight and/or appetite.

As will be readily appreciated, all of the above compounds and/or extracts are commercially available, and it is generally contemplated that these compounds and/or extracts will be used in quantities commonly used in the context of nutritional supplements. Therefore, the quantities of the compounds and/or extracts is preferably at least 10 mg, or at least 100 mg, or at least 200 mg, or at least 300 mg, or at least 400 mg, or at least 500 mg, or at least 750 mg, or at least 1,000 mg, or at least 1,500 mg, or at least 2,000 mg, or at least 2,500 mg, or at least 3,000 mg, or at least 3,500 mg, or at least 4,000 mg, but in most cases below 10,000 mg, or below 8,000 mg, or below 6,000 mg. For example, suitable dosages will be between 10-100 mg, or between 100-200 mg, or between 200-400 mg, or between 300-600 mg, or between 400-800 mg, or between 600-1,000 mg, or between 750-1,500 mg or between 750-2,000 mg, or between 1,500-2,500 mg.

In further contemplated aspects, suitable quantities may also be determined by the synergistic effect of the compounds and/or extracts in combination with the polyphenols and polyphenol mixtures. Therefore, suitable ratios of the compounds and/or extracts to the polyphenols and polyphenol mixtures will be between 1:50 to 1:20, or between 1:20 and 1:10, or between 1:10 and 1:5, or between 1:5 and 1:2, or between 1:2 and 2:1, or between 2:1 and 5:1, or between 5:1 and 10:1, or between 10:1 and 20:1, or between 20:1 and 50.

With respect to the polyphenols and polyphenol mixtures it is generally contemplated that these compounds and mixtures will be found in nutritionally acceptable materials, and particularly in fruit and/or vegetables suitable for human consumption. Most preferably, but not necessarily, such compounds and mixtures may be characteristic of a specific ethnic and/or geographic diet (e.g., Mediterranean diet, Okinawa diet, Loma Linda (Seventh Day Adventist diet), etc.), or the compounds and mixtures may be selected on the basis of their effect on one or more metabolic or cell signaling pathways. Therefore, in some embodiments suitable blends include blends from colored plant materials that belong to a number (e.g., at least two, at least three, or at least four) of different color groups, and particularly contemplated plant materials may have a red color, green color, orange/yellow color, and/or purple/blue color. For example, in a further embodiment of contemplated compositions, polyphenol-containing products and/or extracts can be obtained from red colored source materials that include an apple extract, a pomegranate extract, tomato powder, and beet root; from green colored source materials that include an olive extract, rosemary extract, green coffee bean extract, and kale; from orange/yellow colored source materials that include an onion extract, a ginger extract, a grapefruit extract, and carrot; and from purple/blue colored source materials that include a grape extract, a blueberry extract, currant, and elderberry, and exemplary ingredients and proportions are described in more detail below.

Viewed from a different perspective, contemplated compositions will therefore include a large number of polyphenols that below to at least two, or at least three, or at least four different polyphenolic classes, including organic acids, phenolics, flavonols, flavanols, anthocyanins, chlorogenic acids, betacyanins, etc. As will be readily appreciated, the particular choice of a plant material will depend on the desired (polyphenolic) component in the plant material and its effect on a particular biological system and/or signaling pathway.

Of course, it should also be appreciated that the plant materials (as well as the compounds and extracts) may be provided in various forms, including whole plant materials or portions thereof (e.g., root, fruit, leaves, etc.) in fresh or dried form, juices or macerates from plant materials or portions thereof in fresh or dried form, and aqueous or aqueous/alcoholic extracts and chromatographic fractions of the aforementioned plant materials. Still further, it should be noted that one or more polyphenols of the plant materials may even be provided as purified (natural isolated or synthetic) chemical entities, typically with a chemical purity of at least 90%, or at least 95%, or at least 98%, or at least 99%. However, it should be recognized that in most embodiments the plant materials will be complex mixtures to provide a combination of desired biological effects on a number of distinct molecular entities (e.g., enzymes, receptors, ion channels) where at least some of the biological effects (e.g., at least one, or at least two, or at least three, etc.) are synergistic. Moreover, it is contemplated that the biological effects on the particular molecular entities will also be complementary in biological function. Therefore, and based on the testing and desired targets as described in more detail below, it should be appreciated that the compositions of the inventive subject matter may be formulated to meet a particular need. However, in especially preferred aspects contemplated compositions will inhibit multiple targets (e.g., at least two, at least three, at least four, etc.) in multiple and distinct (e.g., at least two, at least three, at least four, etc.) signaling pathways, alone, or in conjunction with the compounds and extracts having a known or suspected activity.

Consequently, and viewed from a different perspective, it should be appreciated that the mechanism of action of contemplated compositions is not limited to a single specific function (e.g., antioxidant) or limited to a specific chemical category (e.g., vitamins) according to the specific known or suspected activity of a specific compound or extract, but in fact complementarily and synergistically provides multiple biological activities across distinct metabolic and signaling pathways to thereby enhance the known or suspected activity of the specific compound or extract. Thus, the polyphenols and polyphenol mixtures may be viewed as a multi-modal and/or multi-pathway enhancer of the desired activity of specific compounds or extracts.

Consequently, it should be appreciated that the compositions contemplated herein may be advantageously used as a stand-alone product to support various aspects of health and healthy ageing. In this context, it should be noted that the term “support” when used in conjunction with a physiological function or condition is intended to mean prevent decline of one or more components or activities of the component(s) associated with the physiological function or condition, at least partially reverse decline of one or more components or activities of the component(s) associated with the physiological function or condition, maintain normal function of one or more components or activities of the component(s) associated with the physiological function or condition, prevent abnormal overactivity (or over-expression) of one or more components associated with the physiological function or condition, and/or at least partially reverse abnormal overactivity (or over-expression) of one or more components associated with the physiological function or condition. Thus, it should be recognized that the compositions resented herein present a new and different approach in providing health support that reaches well beyond ordinary health support using just the specific compounds or extracts with known or suspected activity.

In further contemplated aspects of the inventive subject matter, it should be appreciated that the compositions presented herein may be formulated in a variety of forms, and particularly preferred formulations include those in combination with a nutritionally or pharmaceutically acceptable carrier, most preferably for oral administration (however, parenteral administration is also expressly contemplated). Therefore, contemplated compositions can be formulated as solid or a liquid product. For example, where contemplated compositions are formulated as a solid product, suitable product forms include single dosage unit formulations such as capsules, tablets, and powders, while other solid formulations include snack bars, gummies, or other edible products onto which the composition is coated (e.g., onto cereal) or into which the composition is mixed or layered (e.g., into chewing gum). In another examples, where contemplated compositions are formulated as a liquid product, suitable product forms include flavored and/or carbonated beverages (e.g., tea, juice), functional beverages (e.g., sports or energy drinks) or infusions, or liquid dairy product (e.g., yoghourt, kefir).

Therefore, contemplated compositions may be provided in bulk, as part of an edible or drinkable product, and/or provided in single dosage units for consumption. Most typically, the daily dosage for contemplated compositions (excluding the carrier) is preferably at least 10 mg, or at least 100 mg, or at least 200 mg, or at least 300 mg, or at least 400 mg, or at least 500 mg, or at least 750 mg, or at least 1,000 mg, or at least 1,500 mg. For example, suitable dosages will be between 10-100 mg, or between 100-200 mg, or between 200-400 mg, or between 300-600 mg, or between 400-800 mg, or between 600-1,000 mg, or between 1,000-2,000 mg. In still further contemplated aspects, a dosage unit will represent a recommended quantity of the compositions contemplated herein. Therefore, a dosage unit may presented as a single item of consumption (e.g., dosage unit is a single capsule or ready-to drink beverage) or as multiple items of consumption (e.g., dosage unit distributed over 2-4 capsules).

Of course, it should be recognized that the compositions according to the inventive subject matter may be administered not only to a human, but also to other non-human mammals, and especially livestock and companion animals (e.g., dogs, cats, horses). Administration will typically be between once daily and three times daily (and in some cases even more) over a period of at least two days, three days, five days, 1 week, 2-4 weeks, 1-3 months, and even longer. Most typically, administration can be performed for a period sufficient to provide at least symptomatic relief of a condition (e.g., pain and swelling associated with inflammation, low energy level, frequent infections, etc.), or prophylactically to avoid or help reduce severity of a health condition.

### Examples

Representative Polyphenol Composition:

As shown in more detail below, the representative polyphenol composition was based on a polyphenol profile typical for the Mediterranean diet. Here, the polyphenol-containing products/extracts can be obtained from source materials characterized by color as follows: Red group: apple extract, pomegranate extract, tomato powder, and beet root; Green group: olive extract, rosemary extract, green coffee bean extract, and kale; Orange/yellow group: onion extract, ginger extract, grapefruit extract, and carrot; and Purple/blue group: grape extract, blueberry extract, currant, and elderberry. Corn starch, silica, and sunflower lecithin were used as processing aids. Relative proportions are shown in Table 1 below.

TABLE 1

| Ingredient | Wt % | Part | Solvent | Standardized to | State |
|---|---|---|---|---|---|
| Grape Extract | 15.75 | Whole fruit | Ethanol | Min 50% total | Powdered |
| | | | | Proanthocyanidins | |
| Apple Extract | 10.00 | Whole fruit | Ethanol/Water | 70% Polyphenols | Powdered |
| Ginger Extract | 10.00 | Root | Ethanol/Water | 5% Gingerol | Powdered |
| Onion Extract | 10.00 | Root bulb | Ethanol/Water | 30% Quercetin | Powdered |
| Pomegranate Extract | 10.00 | Whole fruit | Ethanol | 40% Punicalagins | Powdered |
| Green Coffee Bean | 7.50 | Seed | Ethanol/Water | 50% Chlorogenic | Powdered |
| Extract | | | | Acids | |
| Rosemary Extract | 7.50 | Leaves | Ethanol/Water | 15% Rosmarinic Acid | Powdered |
| Olive Extract | 6.38 | Whole fruit | Water | 12% Hydroxytyrosol | Powdered |
| Maltodextrin | 3.19 | —/— | —/— | —/— | Powdered |
| Blueberry Extract | 2.50 | Whole fruit | Ethanol/Water | 12% Anthocyanins | Powdered |
| Grapefruit Extract | 2.50 | Whole fruit | Ethanol/Water | 90% Naringin | Powdered |
| Kale | 2.50 | Leaves | None | —/— | Powdered |
| Beet Root | 2.45 | Root | None | —/— | Powdered |
| Carrot | 2.45 | Root | None | —/— | Powdered |
| Black Currant | 2.43 | Whole fruit | None | —/— | Powdered |
| Elderberry | 2.43 | Whole fruit | None | —/— | Powdered |
| Tomato | 1.85 | Whole fruit | None | —/— | Powdered |
| Corn Starch | 0.31 | —/— | —/— | —/— | Powdered |
| Silica | 0.25 | —/— | —/— | —/— | Powdered |
| Sunflower Lecithin | 0.03 | —/— | —/— | —/— | Powdered |

Phytochemical HPLC/MS/MS analysis: An HPLC/MS compositional analysis of the exemplary composition above revealed the following ingredients and proportions where the columns in each of Tables 2-8 indicate the analyte ID (co1.1), chemical entity (co1.2), M-H (co1.3), RT (co1.4), peak intensity (co1.5), and MS/MS fragments (co1.6):

TABLE 2
Organic acids

| | | | | | |
|---|---|---|---|---|---|
| 1 | Citric acid | 191.0186 | 2.9 | 5.25E+08 | 102, 111, 129, |
| | | | | | 173 |
| 2 | Malic acid | 133.0130 | 2.2 | 1.26E+08 | 71, 89, 115 |
| 3 | Glucaric acid | 209.0291 | 1.7 | 8.68E+08 | 133, 147, 191 |
| 4 | Gluconic acid | 195.0504 | 1.7 | 4.44E+08 | 99, 129, 159, |
| | | | | | 177 |
| 5 | hydroxy jasmonic | 387.1668 | 22.3 | 2.23E+08 | 59, 163, 207 |
| | acid-O-glucoside | | | | |
| 6 | Azelaic acid | 187.0968 | 28.3 | 1.56E+07 | 125, 169 |
| 5 | Arabinonic acid | 165.0395 | 1.7 | 1.96E+08 | 129, 147, 165 |

TABLE 3
Phenolics

| | | | | | |
|---|---|---|---|---|---|
| 7 | Coumaric acid | 163.0389 | 24.3 | 9.19E+06 | 119, 147 |
| 8 | Coumaric aicd - derv | 295.0460 | 21.1 | 2.45E+06 | 119, 163 |
| 9 | Quinic acid-I | 191.0553 | 1.8 | 4.76E+08 | 173 |
| 10 | Quinic acid-II | 191.0553 | 20.1 | 2.40E+08 | 173 |
| 11 | Caffeic acid | 179.0341 | 21.4 | 1.54E+08 | 135 |
| 12 | Caffeic acid-hexose-I | 341.0869 | 16.6 | 5.02E+06 | 135, 161, 179 |
| 13 | Caffeic acid-hexose-II | 341.0869 | 18.5 | 1.28E+07 | 135, 161, 179 |
| 14 | Caffeic acid-hexose-III | 341.0869 | 19.6 | 1.02E+07 | 135, 179 |
| 15 | Caffeic acid-hexose-IV | 341.0869 | 20.0 | 1.61E+07 | 135, 161, 179 |
| 16 | Ferulic acid | 193.0498 | 25.6 | 4.32E+06 | 134.04, 149.06, 178.03 |
| 17 | Ferulic acid-hexose-I | 355.1028 | 21.7 | 1.97E+07 | 175 |
| 18 | Ferulic acid-hexose-I | 355.1028 | 22.5 | 5.63E+06 | |
| 19 | Ferulic acid-hexose-I | 355.1028 | 23.3 | 6.47E+06 | 134, 149, 175 |
| 20 | Ferulic acid-hexose-I | 355.1028 | 24.9 | 3.24E+06 | 193 |
| 21 | Ferulic acid-hexose-I | 355.1028 | 25.9 | 4.32E+06 | 193 |
| 22 | 3,4-Dihydroxybenzoic acid | 153.0188 | 10.2 | 1.23E+07 | 109 |
| 23 | Gallic acid | 169.0132 | 5.3 | 6.41E+07 | 125 |
| 24 | Rosamric acid | 359.0772 | 28.8 | 1.18E+09 | 135, 131, 179, 197 |
| 25 | Rosamric acid dimer | 719.1618 | 28.8 | 5.48E+08 | 135, 161, 179, 197 |
| 26 | Salvianic acid A | 197.0447 | 9.2 | 7.12E+07 | 123, 135, 179, 180 |
| 27 | Ethyl gallate | 197.0449 | 24.3 | 1.83E+07 | 125, 151, 169 |
| 28 | Ellagic acid | 300.9994 | 25.8 | 1.20E+08 | 229, 257, 283 |
| 29 | Ellagic acid glucoside | 463.0529 | 22.9 | 5.69E+05 | 125, 169, 300.99, 463 |
| 30 | Galloyl-HHDP-glucoside | 633.0737 | 22.7 | 6.10E+07 | 125, 169, 193, 275, 300.99 |
| 31 | Digalloyl-HHDP-glucoside | 785.0867 | 22.3 | 1.92E+06 | 125, 169, 275, 300.99 |
| 32 | 3-Galloylquinic acid | 343.0673 | 5.9 | 1.70E+06 | 169, 191 |
| | (Theogallin) | | | | |
| 33 | 3,4-Dihydroxybenzoic | 153.0183 | 10.2 | 1.23E+07 | 96,109 |
| 34 | 3,4-Dihydroxybenzoic | 153.0545 | 10.3 | 3.62E+08 | 109, 123, 153.0182 |
| 35 | 2,4-Dihydroxybenzoic | 153.0183 | 23.8 | 8.71E+06 | 109 |
| 36 | 2-Hydroxybenzoic acid | 137.0232 | 15.0 | 1.10E+07 | 93 |

TABLE 4
Flavonoids

| | | | | | |
|---|---|---|---|---|---|
| 37 | Naringenin | 271.0604 | 34.4 | 2.96E+07 | 119, 151, 227, 271 |
| 38 | Naringenin-glucoside | 433.1149 | 28.0 | 4.14E+06 | 151, 271 |
| 39 | Naringenin-rhamnoglucoside | 579.1722 | 27.5 | 1.13E+09 | 151, 271, 549 |
| 40 | Naringenin-rhamnoglucoside dimer | 1159.3540 | 27.5 | 5.66E+07 | 151, 271, 459 |
| 41 | Myricetin | 317.0503 | 29.0 | 1.09E+07 | 137, 151, 178 |
| 42 | Myricetin 3-rhamnoside | 463.0894 | 25.8 | 5.61E+06 | 316, 317 |
| 43 | Myricetin 3-glucoside I | 479.0839 | 24.3 | 4.59E+06 | 316, 317 |
| 44 | Myricetin 3-rhamnoside II | 479.0839 | 24.5 | 4.57E+06 | 316, 317 |
| 45 | Phloretin | 273.0769 | 34.5 | 2.41E+08 | 167 |
| 46 | Phloretin -glucoside | 435.1298 | 29.0 | 2.85E+08 | 167, 273 |
| 47 | Phloretin-arabinose-glucoside | 567.1733 | 27.5 | 3.28E+08 | 167, 273 |
| 48 | Phloretin-diglucoside | 597.1831 | 26.8 | 7.70E+06 | 167, 273 |
| 49 | Apigenin | 269.0460 | 34.4 | 4.46E+07 | 151 |
| 50 | Apigenin glucoside | 431.0985 | 27.8 | 1.36E+07 | 268, 269 |
| 51 | Apigenin rhamnoside glucoside | 577.1575 | 27.4 | 8.96E+07 | 269 |
| 52 | Apigenin rhamnoside glucoside der | 623.1635 | 27.4 | 7.88E+06 | 269, 577 |
| 53 | Apigenin rhamnoside glucoside dimer | 1155.3230 | 27.4 | 1.41E+06 | 269, 577 |
| 54 | Quercetin | 301.0348 | 32.1 | 1.89E+09 | 151, 179, 273, 301 |
| 55 | Quercetin dimer | 603.0795 | 32.1 | 3.94E+08 | 151, 178, 301 |
| 56 | Quercetin galactoside | 463.0874 | 24.6 | 1.00E+07 | 301 |
| 57 | Quercetin glucoside-1 | 463.0874 | 25.9 | 2.98E+07 | 151, 300, 301 |
| 58 | Quercetin glucoside-2 | 463.0874 | 26.1 | 7.16E+07 | 151, 300, 301 |
| 59 | Quercetin-diglucoside | 625.1400 | 23.8 | 8.83E+05 | 151, 178, 301, 463 |
| 60 | Quercetin-rutinoside or Rutin | 609.1478 | 25.5 | 3.37E+07 | 300, 301 |
| 61 | Luteolin | 285.0407 | 31.9 | 4.20E+07 | 133.03, 199.04, |
| | | | | | 217.05, 241 |
| 62 | Luteolin glucoside 1 | 447.0938 | 26.2 | 4.58E+06 | 284, 285 |
| 63 | Luteolin glucoside 2 | 447.0938 | 24.6 | 1.50E+07 | 284, 285 |
| 64 | Catechin-1 | 289.0704 | 20.2 | 5.97E+08 | 151, 179, 205, 245 |
| 65 | Catechin-2 | 289.0704 | 22.7 | 8.47E+08 | |
| 66 | Proanthocyanidin B1 | 577.1364 | 22.0 | 4.06E+08 | 125, 289, 407 |
| 67 | Proanthocyanidin B2 | 577.1364 | 18.9 | 2.14E+08 | 125, 289, 407 |
| 68 | Isorhamnetin | 315.0503 | 35.4 | 2.66E+08 | 300, 315 |
| 69 | Isorhamnetin glucoside | 477.1030 | 26.7 | 1.11E+08 | 119, 151, 299, 314, |
| | | | | | 315 |
| 70 | Isorhamnetin diglucoside-1 | 639.1563 | 27.4 | 1.11E+06 | 151, 285, 313, 315, |
| | | | | | 476, 477, 639 |
| 71 | Isorhamnetin-rhamnosy1-glucoside | 623.1939 | 26.1 | 6.63E+06 | 315 |
| 72 | Kaempferol | 285.0397 | 35.0 | 1.27E+08 | 125.02, 244, 257, 268 |
| 73 | Kaempferol glucoside-1 | 447.0938 | 26.9 | 4.02E+06 | |
| 74 | Kaempferol glucoside-2 | 447.0938 | 27.4 | 1.98E+07 | |
| 75 | Kaempferol glucoside-3 | 447.0938 | 27.6 | 1.04E+07 | |
| 76 | Kaempferol glucoside-4 | 447.0938 | 27.9 | 4.47E+06 | |
| 77 | Kaempferol-3-O-rutinoside | 593.1521 | 25.6 | 1.68E+07 | 285 |
| 78 | Kampferol 3-(6-glucosylglucoside) | 755.2060 | 25.9 | 1.23E+06 | 285 |
| | 7-rhamnoside | | | | |
| 79 | wogonin | 283.0616 | 41.2 | 1.42E+08 | 268, 283 |

TABLE 5
Anthocyanins

| | | | | | |
|---|---|---|---|---|---|
| 80 | Cyanidin-3-O-glucoside | 449.1073 | 21.1 | 1.67E+07 | 287 |
| 81 | Cyanidin derv | 463.0874 | 27.7 | 3.56E+05 | 287 |
| 82 | Cyanidin derv | 463.0874 | 29.1 | 1.97E+07 | 287 |
| 83 | Cyanidin-3-O-rutinoside | 595.1658 | 25.6 | 8.57E+05 | 287, 449 |
| 84 | Cyanidin 3-sambubioside | 581.1500 | 21.1 | 5.95E+06 | 287 |
| 85 | Pelargonidin | 271.0600 | 24.5 | 2.10E+06 | 271 |
| 86 | Pelargonidin-glucoside | 433.1131 | 27.9 | 7.85E+05 | 271 |
| 87 | Pelargonidin-glucoside derv | 639.1710 | 31.4 | 5.30E+05 | 175, 207, 271 |
| 88 | Malvidin | 331.0809 | 26.7 | 2.27E+06 | 316 |
| 89 | Malvidin arabinoside | 463.1237 | 23.0 | 8.98E+06 | 331 |
| 90 | Malvidin-feruloyl-arabinoside | 639.1710 | 28.1 | 7.34E+05 | 331 |
| 91 | Delphinidin | 303.0496 | 32.2 | 2.73E+07 | 303 |
| 92 | Delphinidin 3-glucoside | 465.1028 | 20.1 | 6.49E+06 | 303, 385 |
| 93 | Delphinidin 3-rutinoside | 611.1603 | 25.5 | 1.30E+06 | 303 |
| 94 | Delphinidin-3-arabinoside-I | 435.0922 | 20.7 | 2.06E+06 | 303 |
| 95 | Petunidin | 317.0655 | 35.5 | 2.77E+06 | 317 |
| 96 | Petunidin 3-glucoside | 479.1186 | 26.7 | 1.32E+07 | 317 |
| 97 | Petunidin 3-rutinoside | 625.1765 | 26.1 | 5.63E+05 | 317 |
| 98 | Petunidin derv | 463.0874 | 26.9 | 4.03E+05 | 317 |
| 99 | Peonidin | 301.0709 | 34.8 | 1.63E+06 | 286 |
| 100 | Peonidin-glucoside-I | 463.1240 | 26.9 | 2.77E+06 | 301 |
| 101 | Peonidin-glucoside II | 463.1240 | 28.3 | 6.98E+06 | 301 |
| 102 | Peonidin-rutinoside | 609.1814 | 27.6 | 1.52E+06 | 301 |
| 103 | Peonidin-feruloyl-glucoside | 639.1710 | 31.7 | 1.88E+06 | 177, 301 |

TABLE 6
Chlorogenic acids

| | | | | | |
|---|---|---|---|---|---|
| 104 | 3-Caffeoylquinic acid | 353.0875 | 14.3 | 2.70E+08 | 135, 179, 191 |
| 105 | 5-Caffeoylquinic acid | 353.0875 | 20.1 | 8.52E+08 | 179, 191 |
| 106 | 4-Caffeoylquinic acid | 353.0875 | 21.1 | 5.09E+08 | 135, 173, 179, 191 |
| 107 | 3-Caffeoylquinic acid dimer | 707.1830 | 14.3 | 1.87E+07 | 135, 179, 191, 353 |
| 108 | 5-Caffeoylquinic acid dimer | 707.1833 | 20.1 | 9.23E+08 | 191, 353 |
| 109 | 4-Caffeoylquinic acid dimer | 707.1834 | 21.1 | 1.95E+08 | 135, 173, 179, 191, 353 |
| 110 | 3-Caffeoylshikimic acid | 335.0770 | 24.2 | 3.17E+07 | 135, 161, 173, 179 |
| 111 | 4-Caffeoylshikimic acid | 335.0770 | 24.5 | 1.62E+08 | 135, 161 |
| 112 | 5-Caffeoylshikimic acid | 335.0770 | 25.0 | 1.53E+08 | 161 |
| 113 | 3,4-Dicaffeoylshikimic acid | 497.1091 | 29.5 | 0 | |
| 114 | 3,5-Dicaffeoylshikimic acid | 497.1091 | 32.0 | 9.10E+07 | 161, 179 335 |
| 115 | 4,5-Dicaffeoylshikimic acid | 497.1091 | 32.7 | 6.97E+06 | 135, 161, 179, 335 |
| 116 | 3-Feruloylquinic acid | 367.1032 | 20.7 | 1.13E+08 | 134, 193 |
| 117 | 5-Feruloylquinic acid | 367.1032 | 23.6 | 3.76E+08 | 163, 191 |
| 118 | 4-Feruloylquinic acid | 367.1035 | 23.8 | 1.66E+08 | 173, 193 |
| 119 | 3-Feruloylquinic acid dimer | 735.2144 | 20.7 | 7.14E+05 | |
| 120 | 5-Feruloylquinic acid dimer | 735.2144 | 23.6 | 1.03E+08 | 173, 191, 367 |
| 121 | 4-Feruloylquinic acid dimer | 735.2144 | 23.8 | 4.17E+06 | 173, 193, 367 |
| 122 | 3-Coumaroylquinic acid | 337.0932 | 19.0 | 2.68E+07 | 119, 163 |
| 123 | 4-Coumaroylquinic acid | 337.0933 | 22.6 | 9.95E+07 | 191 |
| 124 | 5-Coumaroylquinic acid | 337.0933 | 23.0 | 4.09E+08 | 163, 173 |
| 125 | 3-Coumaroylquinic acid dimer | 675.1941 | 19.0 | 0 | |
| 126 | 4-Coumaroylquinic acid dimer | 675.1941 | 22.6 | 3.00E+06 | 191 |
| 127 | 5-Coumaroylquinic acid dimer | 675.1941 | 23.0 | 1.53E+07 | 163, 173 |
| 128 | 3,4-Dicaffeoyl quinic acid | 515.1189 | 27.1 | 2.63E+08 | 135, 161, 173, 179, 191, |
| | | | | | 353 |
| 129 | 3,5-Dicaffeoyl quinic acid | 515.1190 | 27.6 | 1.79E+08 | 135, 179, 191, 353 |
| 130 | 4,5-Dicaffeoyl quinic acid | 515.1195 | 28.3 | 3.37E+08 | 135, 173, 179, 191, 353 |
| 131 | Valeroylquinic acid isomer-1 | 275.1135 | 14.9 | 2.70E+05 | 101, 173, 181 |
| 132 | Valeroylquinic acid isomer-2 | 275.1135 | 15.8 | 5.75E+05 | 101, 181, 191 |
| 133 | Valeroylquinic acid isomer-3 | 275.1135 | 21.0 | 1.17E+06 | 101, 173 |
| 134 | Valeroylquinic acid isomer-4 | 275.1135 | 21.4 | 2.26E+06 | 101, 173 |
| 135 | Valeroylquinic acid isomer-5 | 275.1135 | 23.2 | 3.27E+05 | 93, 101, 173, 181, 191 |
| 136 | Valeroylquinic acid isomer-6 | 275.1135 | 23.4 | 5.25E+05 | 101, 173 |
| 137 | Valeroylquinic acid isomer-7 | 275.1135 | 30.4 | 5.22E+06 | 93, 101, 173, 181, 191 |
| 138 | Valeroylquinic acid isomer-8 | 275.1135 | 30.6 | 1.18E+06 | 101 |
| 139 | Valeroylquinic acid isomer-9 | 275.1135 | 31.7 | 1.98E+06 | 101, 173 |
| 140 | Caffeoylvaleroylquinic acid isomer 1 | 437.1451 | 29.7 | 3.55E+06 | 161, 173, 179, 275 |
| 141 | Caffeoylvaleroylquinic acid isomer 2 | 437.1451 | 29.8 | 6.88E+06 | 161, 173, 179, 275 |
| 142 | Caffeoylvaleroylquinic acid isomer 3 | 437.1451 | 30.4 | 2.46E+06 | 161, 173, 179, 275 |
| 143 | Caffeoylvaleroylquinic acid isomer 4 | 437.1451 | 30.6 | 5.13E+06 | 173, 275 |
| 144 | Caffeoylvaleroylquinic acid isomer 5 | 437.1451 | 31.7 | 6.96E+06 | 173, 275 |
| 145 | Quinic acid-glucoside-R*-1 | 481.2442 | 24.8 | 3.81E+07 | 161, 197, 301 |
| 146 | Quinic acid-glucoside-R*-2 | 481.2442 | 25.3 | 1.22E+08 | 161, 197, 301 |
| 147 | Valeroylquinic acid glucoside-R*-1 | 565.3023 | 33.3 | 1.18E+07 | 301, 463, 481 |
| 148 | Valeroylquinic acid diglucoside-R*-1 | 727.3545 | 26.6 | 3.83E+06 | 161, 301, 323, 481, 643 |
| 149 | Valeroylquinic acid diglucoside-R*-2 | 727.3545 | 30.4 | 1.89E+06 | 205, 361, 625, 643 |
| 150 | Valeroylquinic acid diglucoside-R*-3 | 727.3545 | 30.8 | 3.05E+07 | 481, 523, 625, 643 |

TABLE 7
Betacyanins

| | | | | |
|---|---|---|---|---|
| 151 | Betanin | 551.1517 | 15.6 | 2.19E+04 |
| 152 | Isobetanin | 551.1517 | 19.7 | 1.54E+04 |

TABLE 8
Amino acids, alkaloids & other compounds

| | | | | | |
|---|---|---|---|---|---|
| 153 | Glycerophosphocholine | 258.1098 | 1.7 | 6.17E+07 | 104 |
| 154 | Adenosine | 268.1040 | 4.1 | 4.68E+06 | 136 |
| 155 | Phenylalanine | 166.0861 | 6.8 | 1.41E+07 | 120 |
| 156 | Tryptophan | 205.0973 | 14.5 | 6.28E+06 | 146, 188 |
| 157 | Tyrosine | 182.0812 | 3.7 | 9.16E+06 | 119, 123, 136, 147, 165 |
| 158 | Dopamine | 154.0858 | 2.7 | 8.64E+05 | 113, 137 |
| 159 | Trigonellin | 138.0548 | 1.8 | 2.05E+08 | 94 |
| 160 | Caffeine | 195.0875 | 20.1 | 4.94E+07 | 138 |
| 161 | Xanthine | 151.0256 | 3.1 | 7.74E+05 | 109 |

Representative Compounds And Extracts Having Known Or Suspected Activity:

As shown in more detail below, a variety of compounds and extracts can be used for combination with the (representative) polyphenol composition, and Table 9 exemplarily lists selected compounds, extracts, and quantities suitable for use herein.

TABLE 9

| Known or | |
|---|---|
| Suspected Activity | Compounds/Extracts |
| Bone and Joint | Vitamin D3, K2, B3, A |
| | Green tea catechins |
| | Chondroitin |
| | Glucosamine |
| | Ca Fructoborate |
| | MSM |
| | Collagen II |
| | Hyaluronic acid |
| | L-Lysine |
| | Eggshell membrane |
| | Ca, Mg, Zn, Cu, Sr |
| | Omega-3s |
| | Turmeric |
| | Curcumin |
| | Boswellia |
| | Demineralized bone matrix |
| Skin and Hair | Alpha Lipoic Acid |
| | MSM |
| | Collagen |
| | Keratin |
| | Cysteine |
| | Aloe vera |
| | Turmeric |
| | Ginger |
| | Omega-3s |
| | PUFAs |
| | CoQ10 |
| | Biotin |
| | Mg, Se, Zn, Cu, Fe, Si |
| | Vitamin B3/6, D, E Biotin |
| | Hyaluronic acid |
| | Hydroxyproline |
| | Ceramides |
| | Horsetail grass |
| Cellular Energy | CoQ10 |
| | MCT |
| | Ribose |
| | Niacin |
| | Nicotinamide |
| | Nicotinamide riboside |
| | NAD |
| Inflammation | Omega-3s |
| | MSM |
| | Melatonin |
| | Green tea catechins |
| | Ursolic acid |
| | Resveratrol |
| | Lignans (sesamine, etc) |
| Immunity | Omega-3s |
| | Zn |
| | N-acetylcysteine |
| | Mushroom polysaccharidesTurkey |
| | tail, lion's mane, shiitake, cordyceps, |
| | reishi, etc.) |
| | Elderberry |
| | Vitamin C |
| | Echinacea |
| | Garlic extract |
| | Probiotics |
| | Colostrum |
| | Beta glucans |
| | Grapefruit seed extract |
| | Oscillococcinum |
| | Inositol hexaphosphate |
| | Lignans (sesamin) |
| Stress | SAM |
| | Ashwagandha |
| | 5-HTP |
| | Theanine |
| | GABA |
| | Kava extract |
| | Valerian extract |
| | Mg |
| | Vitamin B6, B12 |
| | N-acetyltrosine |
| | St. John's wort |
| Sleep | Omega-3s |
| | Melatonin |
| | Hops |
| | Chamomile |
| | Valerian extract |
| | Passion flower extract |
| | St. John's wort |
| Heart | CoQ10 |
| | Omega-3s |
| | MSM |
| | Red yeast rice |
| | Phytosterols (beta sitosterol) |
| | Polycosanol |
| | Niacin |
| | Garlic extract |
| | Hawthorn extract |
| Weight/Appetite | Alpha Lipoic Acid |
| | MCFA |
| | SCFA |
| | CLA |
| | Ketones (BHB, ACAC . . . ) |
| | Caffeine |
| | Theanine |
| | Carnitine |
| | Green tea catechins |
| | Ursolic acid |
| | Resveratrol |
| | Quercetin |
| | Yohimbe bark extract |
| Men's | Fenugreek extract |
| | Saw palmetto extract |
| | Pumpkin seed extract |
| | Nettle extract |
| | Ginger extract |
| | Green tea catechins |
| | Lycopene |
| | DHEA |
| | Phytosterols |
| | Turmeric extract |
| | Pygeum bark extract |
| | Ca, Zn, Se, Cu, Mb, Mn, B |
| | Yohimbine |
| | Ginkgo extract |
| | Ginseng extract |
| | Tribulus extract |
| | Horny goat extract |
| | Maca root extract |
| | Tongkat Ali extract |
| | Vitamin E, B6, B12 |
| Woman's | DHEA |
| | Omega-3s |
| | Vitamin B1, B3, B6, B12, C, D3 |
| | CoQ10 |
| | Biotin |
| | Folate |
| | Probiotics |
| | Cranberry extract |
| | Mannose |
| | Black cohosh extract |
| | Soy isoflavones |
| | Phytoestrogens |
| | Wild yam extract |
| | Chasteberry extract |
| | Cissus quadrangularis extract |
| | Licorice root extract |
| | Dong Quai root extract |
| | Maca root extract |
| Mind/Brain | Omega-3s |
| | Lecithin |
| | Phosphatidylserine |
| | Creatine |
| | Theanine |
| | Tyrosine |
| | N-acetyl-carnitine |
| | N-acetylcysteine |
| | N-acetyltyrosine |
| | Cytidine 5-phosphocholine |
| | Caffeine |
| | Coffee cherry extract |
| | Ginkgo biloba |
| | Alpha Lipoic Acid |
| | Apoaequorin |
| | Uridine-5-monophosphae |
| Mood | DHEA |
| | SAM |
| | EPA (Eicosapentaenoic Acid) |
| | Bacopa monierii |
| | Caffeine |
| | 5-HTP |
| | Typotophan |
| | St. John's wort |
| Antioxidant | Alpha Lipoic Acid |
| | MSM |
| | Melatonin |
| | Green tea catechins |
| | Resveratrol |
| | Rhodiola extract |
| Digestive | Alpha Lipoic Acid |
| | Prebiotics (FOS, XOS, etc.) |
| | Probiotics |
| | Bromelain |
| | Papain |
| | Ginger |
| | Amylase |
| | Lipase |
| | Lactase |
| | Galctosidase |
| | Xylanase |
| | SCFA |
| | N-acetylcysteine |
| | Milk Thistle Extract |
| | Fibers |
| Strength/Fitness | CoQ10 |
| | Ketones |
| | Creatine |
| | Betalains |
| | Plant and whey protein |

Aspects

The present disclosure will be better understood upon reading the following numbered aspects, which should not be confused with the claims. In some instances, each of the aspects described below can be combined with other aspects, including combined with other aspects described elsewhere in the disclosure or other aspects from the examples below, without departing from the spirit of the disclosure.
1. An enhanced nutritional composition, comprising a nutritionally acceptable compound or extract having known or suspected activity; a plurality of chemically distinct polyphenol-containing plant materials or chemically distinct polyphenols for plant materials; wherein the nutritional composition is formulated for oral administration; and wherein the plant materials or chemically distinct polyphenols are present in the nutritional composition in an amount effective in increase the activity as compared to the compound or extract alone.
2. The composition of aspect 1, wherein the plant materials have a red color, a green color, an orange-yellow color, and a purple-blue color.
3. The composition of aspect 2, wherein the (a) red colored plant materials comprise an apple extract, a pomegranate extract, a tomato powder, and a beet root powder; (b) green colored plant materials comprise an olive extract, a rosemary extract, a green coffee bean extract, and a kale powder; (c) orange-yellow colored plant materials comprise an onion extract, a ginger extract, a grapefruit extract, and a carrot powder; and (d) purple-blue colored plant materials comprise a grape extract, a blueberry extract, a currant powder, and an elderberry powder.
4. The composition of any one of aspects 1-3, wherein the nutritionally acceptable compound or extract is selected from the group consisting of a vitamin, an isolated or purified compound, an element in ionic form, a complex organic molecule of biological structure, a plant part or plant extract, an enzyme, and a probiotic.
5. The composition of aspect 4, wherein the vitamin is selected from the group consisting of vitamin A, vitamin B1, vitamin B3 (niacin) or derivative or metabolite thereof, vitamin B6, vitamin B7 (biotin), vitamin B9 (folate), vitamin B12, vitamin C, vitamin D3, vitamin E, CoQ10, and vitamin K2.
6. The composition of aspect 4, wherein the isolated or purified compound is selected form the group consisting of 5-gamma-aminobutyric acid (GABA), alpha lipoic acid (ALA), apoaequorin, a betalain, caffeine, carnitine, a ceramide, chondroitin, a conjugated linoleic acid (CLA), creatine, curcumin, cysteine, a green tea catechin (EGCG, EC, EGC, GCG, GC), cytidine 5-phosphocholine, dehydroepiandrosterone (DHEA), eicosapentaenoic Acid (EPA), a medium-chain fatty acid (MCFA), a short chain fatty acid (SCFA), an omega-3 fatty acid, a monounsaturated fatty acid (MUFA, and especially oleic acid), a polyunsaturated fatty acid (PUFA), a fructoborate, Glucosamine, hyaluronic acid, hydroxyproline, hydroxytryptophan (5-HTP), a soy isoflavone, inositol hexaphosphate, a ketone, a lignan, lecithin, lycopene, lysine, mannose, melatonin, methylsulfonylmethane (MSM), N-acetyltrosine, theanine, a phytosterol, N-acetyl-carnitine, N-acetylcysteine, N-acetyltyrosine, phosphatidylserine, a phytoestrogen, a polycosanol, quercetin, resveratrol, ribose, S-adenosylmethionine (SAM), Tryptophane, tyrosine, uridine-5-monophosphate, ursolic acid, and yohimbine.
7. The composition of aspect 4, wherein the element in ionic form is selected from the group consisting of boron, calcium, copper, iron, molybdenum, magnesium, manganese, selenium, silicon, strontium, and zinc.
8. The composition of aspect 4, wherein the complex organic molecules or biological structure is selected for the group consisting of collagen (esp. type II), colostrum, a demineralized bone matrix, eggshell membrane, a fiber, a beta glucan, a mushroom polysaccharide, a plant or whey protein, a prebiotic, and a keratin.
9. The composition of aspect 4, wherein the plant part and/or extract from a plant is selected from a plant of the group consisting of Aloe vera, Ashwagandha spec., Bacopa monierii, Black cohosh, Boswellia spec., Chamomile, Chasteberry, Cissus quadrangularis, Coffee cherry, Cranberry, Dong Quai, Echinacea, Elderberry, Fenugreek, Garlic, Ginger, Ginkgo biloba, Ginseng, Grapefruit, Hawthorn, Horny goat, Horsetail grass, Hops, Kava, Licorice, Maca, Milk Thistle, Nettle, St. John's wort, Tongkat Ali, Passion flower, Pumpkin, Pygeum, Red yeast rice, Rhodiola, Saw palmetto, Tribulus, Turmeric, Valerian, Wild yam, and Yohimbe.
10. The composition of aspect 4, wherein the enzyme is selected from the group consisting of an amylase, bromelain, a galactosidase, papain, a lactase, a lipase, and a xylanase.
11. The composition of aspect 4, wherein the probiotic is selected form the group consisting of Lactobacillus acidophilus, Lactobacillus casei, Lactobacillus plantarum, Lactobacillus brevis, Lactobacillus gasseri, Lactobacillus rhamnosus, Bifidobacterium lactis, Bifidobacterium breve, and Bifidobacterium longum.
12. The composition of any one of aspects 1-11, wherein the known or suspected activity is selected form the group consisting of support of bone and joint health, support of hair and skin health, support of cellular energy, reduction in inflammation, support of immunity, reduction in stress, support of sleep health, support of heart health, reduction of weight and/or appetite, support of men's health, support of woman's health, support of brain and/or mind health, support of mood, support of digestive health, and support of strength and/or fitness.
13. The composition of any one of aspects 1-12, wherein the known or suspected activity is support of bone and joint health, and wherein the nutritionally acceptable compound or extract is selected from the group consisting of vitamin D3, vitamin K2, vitamin B3, vitamin A, a green tea catechin, chondroitin, glucosamine, calcium fructoborate, methylsulfonylmethane (MSM), Collagen type II, a hyaluronic acid, L-lysine, an eggshell membrane, calcium, magnesium, zinc, copper, strontium, an omega-3 fatty acid, a turmeric portion or extract thereof, curcumin, a Boswellia portion or extract thereof, and a demineralized bone matrix.
14. The composition of any one of aspects 1-12, wherein the known or suspected activity is support of hair and skin health, and wherein the nutritionally acceptable compound or extract is selected from the group consisting of an alpha lipoic acid, methylsulfonylmethane (MSM), a collagen, keratin, L-cysteine, an Aloe vera portion or extract thereof, a turmeric portion or extract thereof, a ginger portion or extract thereof, an omega-3 fatty acid, a monounsaturated fatty acid (MUFA, and especially oleic acid), a polyunsaturated fatty acid (PUFA), CoQ10, biotin, magnesium, selenium, zinc, copper, iron, silicon, vitamin B3, vitamin B6, vitamin D, vitamin E, biotin, a hyaluronic acid, hydroxyproline, a ceramide, and a Horsetail grass portion or extract thereof.
15. The composition of any one of aspects 1-12, wherein the known or suspected activity is support of cellular energy, and wherein the nutritionally acceptable compound or extract is selected from the group consisting of CoQ10, a medium chain fatty acid (MCT), a ribose, niacin, nicotinamide, nicotinamide riboside, and NAD.
16. The composition of any one of aspects 1-12, wherein the known or suspected activity is reduction in inflammation, and wherein the nutritionally acceptable compound or extract is selected from the group consisting of an omega-3 fatty acid, methylsulfonylmethane (MSM), melatonin, a green tea catechin, ursolic acid, resveratrol, and a lignan.
17. The composition of any one of aspects 1-12, wherein the known or suspected activity is support of immunity, and wherein the nutritionally acceptable compound or extract is selected from the group consisting of an omega-3 fatty acid, Zn+, N-acetylcysteine, a mushroom polysaccharide, an Elderberry portion or extract thereof, Vitamin C, an Echinacea portion or extract thereof, a Garlic portion or extract thereof, a probiotic, colostrum, a beta glucan, a Grapefruit portion or extract thereof, Oscillococcinum, inositol hexaphosphate, and a lignan.
18. The composition of any one of aspects 1-12, wherein the known or suspected activity is reduction in stress, and wherein the nutritionally acceptable compound or extract is selected from the group consisting of S-adenosylmethionine (SAM), an Ashwagandha portion or extract thereof, 5-hydroxytryptophane (5-HTP), theanine, gamma-aminobutyric acid (GABA), a Kava portion or extract thereof, a Valerian portion or extract thereof, Mg2+, vitamin B6, vitamin B12, N-acetyltrosine, and a St. John's wort portion or extract thereof.
19. The composition of any one of aspects 1-12, wherein the known or suspected activity is support of sleep health, and wherein the nutritionally acceptable compound or extract is selected from the group consisting of an omega-3 fatty acid, melatonin, a Hops portion or extract thereof, a Chamomile portion or extract thereof, a Valerian portion or extract thereof, a Passion flower portion or extract thereof, and a St. John's wort portion or extract thereof.
20. The composition of any one of aspects 1-12, wherein the known or suspected activity is support of heart health, and wherein the nutritionally acceptable compound or extract is selected from the group consisting of CoQ10, an omega-3 fatty acid, methylsulfonylmethane (MSM), red yeast rice, a phytosterol, a polycosanol, Niacin, a Garlic portion or extract thereof, and a Hawthorn portion or extract thereof.
21. The composition of any one of aspects 1-12, wherein the known or suspected activity is reduction of weight and/or appetite, and wherein the nutritionally acceptable compound or extract is selected from the group consisting of an alpha lipoic acid, a medium chain fatty acid (MCFA), a short chain fatty acid (SCFA), a conjugated linoleic acid (CLA), a ketone, caffeine, theanine, carnitine, a green tea catechin, ursolic acid, resveratrol, quercetin, and a Yohimbe portion or extract thereof
22. The composition of any one of aspects 1-12, wherein the known or suspected activity is support of men's health, and wherein the nutritionally acceptable compound or extract is selected from the group consisting of a Fenugreek portion or extract thereof, a Saw palmetto portion or extract thereof, a Pumpkin portion or extract thereof, a Nettle portion or extract thereof, a Ginger portion or extract thereof, a green tea catechin, lycopene, dehydroepiandrosterone (DHEA), a phytosterol, a Turmeric portion or extract thereof, a Pygeum portion or extract thereof, calcium, zinc, selenium, copper, molybdenum, manganese, boron, Yohimbine, a Ginkgo portion or extract thereof, a Ginseng portion or extract thereof, a Tribulus portion or extract thereof, a Horny goat portion or extract thereof, a Maca portion or extract thereof, a Tongkat Ali portion or extract thereof, vitamin E, vitamin B6, and vitamin B12.
23. The composition of any one of aspects 1-12, wherein the known or suspected activity is support of woman's health, and wherein the nutritionally acceptable compound or extract is selected from the group consisting of an omega-3 fatty acid, vitamin B1, vitamin B3, vitamin B6, vitamin B7 (biotin), vitamin B9 (folate), vitamin B12, vitamin C, vitamin D3, CoQ10, a probiotic, a Cranberry portion or extract thereof, mannose, a Black cohosh portion or extract thereof, a soy isoflavone, a phytoestrogen, a Wild yam portion or extract thereof, a Chasteberry portion or extract thereof, a Cissus quadrangularis portion or extract thereof, a Licorice portion or extract thereof, a Dong Quai portion or extract thereof, and Maca portion or extract thereof
24. The composition of any one of aspects 1-12, wherein the known or suspected activity is support of brain and/or mind health, and wherein the nutritionally acceptable compound or extract is selected from the group consisting of an omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a Coffee fruit portion or extract thereof, a Ginkgo biloba portion or extract thereof, an alpha lipoic acid, apoaequorin, and uridine-5-monophosphate.
25. The composition of any one of aspects 1-12, wherein the known or suspected activity is support of mood, and wherein the nutritionally acceptable compound or extract is selected from the group consisting of dehydroepiandrosterone (DHEA), S-adenosylmethionine (SAM), eicosapentaenoic acid (EPA), a Bacopa monierii portion or extract thereof, caffeine, 5-hydroxytryptophane (5-HTP), tryptophan, and a St. John's wort portion or extract thereof.
26. The composition of any one of aspects 1-12, wherein the known or suspected activity is support of digestive health, and wherein the nutritionally acceptable compound or extract is selected from the group consisting of an alpha lipoic acid, a prebiotic, a probiotic, bromelain, papain, a Ginger portion or extract thereof, an amylase, a lipase, a lactase, a galactosidase, a xylanase, a short chain fatty acid (SCFA), N-acetylcysteine, a Milk Thistle portion or extract thereof, a soluble fiber, and an insoluble fiber.
27. The composition of any one of aspects 1-12, wherein the known or suspected activity is support of strength and/or fitness, and wherein the nutritionally acceptable compound or extract is selected from the group consisting of CoQ10, a ketone, creatine, a betalains, a plant protein, and a whey protein.
28. The composition of any one of aspects 1-27, wherein the composition is formulated in single dosage units for oral administration.
29. The composition of aspect 28, wherein the dosage unit contains between 50 and 1,000 mg of the composition.
30. The composition of aspect 28, wherein the dosage unit is formulated as a capsule, a gummy, or a powder.
31. An enhanced nutritional composition, comprising: a nutritionally acceptable compound or extract having a known or suspected activity selected from the group consisting of support of bone and joint health, support of hair and skin health, support of cellular energy, reduction in inflammation, support of immunity, reduction in stress, support of sleep health, support of heart health, reduction of weight and/or appetite, support of men's health, support of woman's health, support of brain and/or mind health, support of mood, support of digestive health, and support of strength and/or fitness; and a plurality of chemically distinct polyphenol-containing plant materials or chemically distinct polyphenols from plant materials, wherein the plant materials have a red color, a green color, an orange-yellow color, and a purple-blue color.
32. The enhanced nutritional composition of aspect 31, wherein the (a) red colored plant materials comprise an apple extract, a pomegranate extract, a tomato powder, and a beet root powder; (b) green colored plant materials comprise an olive extract, a rosemary extract, a green coffee bean extract, and a kale powder; (c) orange-yellow colored plant materials comprise an onion extract, a ginger extract, a grapefruit extract, and a carrot powder; and (d) purple-blue colored plant materials comprise a grape extract, a blueberry extract, a currant powder, and an elderberry powder.
33. The enhanced nutritional composition of any one of aspect 31 or 32, wherein the composition is formulated in single dosage units for oral administration.
34. The enhanced nutritional composition of aspect 33, wherein the dosage unit contains between 50 and 1,000 mg of the composition.
35. The composition of aspect 33, wherein the dosage unit is formulated as a capsule, a gummy, or a powder.
36. The composition of aspect 33, wherein a dosage unit contains vitamin D3, vitamin K2, vitamin B3, vitamin A, a green tea catechin, chondroitin, glucosamine, calcium fructoborate, methylsulfonylmethane (MSM), Collagen type II, a hyaluronic acid, L-lysine, an eggshell membrane, calcium, magnesium, zinc, copper, strontium, an omega-3 fatty acid, a turmeric portion or extract thereof, curcumin, a Boswellia portion or extract thereof, and/or a demineralized bone matrix as the compound or extract and, upon oral administration to a subject, supports bone and joint health in the subject.
37. The composition of aspect 33, wherein a dosage unit contains alpha lipoic acid, methylsulfonylmethane (MSM), a collagen, keratin, L-cysteine, an Aloe vera portion or extract thereof, a turmeric portion or extract thereof, a ginger portion or extract thereof, an omega-3 fatty acid, a monounsaturated fatty acid (MUFA, and especially oleic acid), a polyunsaturated fatty acid (PUFA), CoQ10, biotin, magnesium, selenium, zinc, copper, iron, silicon, vitamin B3, vitamin B6, vitamin D, vitamin E, biotin, a hyaluronic acid, hydroxyproline, a ceramide, and/or a Horsetail grass portion or extract thereof as the compound or extract and, upon oral administration to a subject, supports hair and skin health in the subject.
38. The composition of aspect 33, wherein a dosage unit contains of CoQ10, a medium chain fatty acid (MCT), a ribose, niacin, nicotinamide, nicotinamide riboside, and/or NAD as the compound or extract and, upon oral administration to a subject, supports cellular energy in the subject.
39. The composition of aspect 33, wherein a dosage unit contains omega-3 fatty acid, methylsulfonylmethane (MSM), melatonin, a green tea catechin, ursolic acid, resveratrol, and/or a lignan as the compound or extract and, upon oral administration to a subject, reduces inflammation in the subject.
40. The composition of aspect 33, wherein a dosage unit contains omega-3 fatty acid, Zn+, N-acetylcysteine, a mushroom polysaccharide, an Elderberry portion or extract thereof, Vitamin C, an Echinacea portion or extract thereof, a Garlic portion or extract thereof, a probiotic, colostrum, a beta glucan, a Grapefruit portion or extract thereof, Oscillococcinum, inositol hexaphosphate, and/or a lignan as the compound or extract and, upon oral administration to a subject, supports immunity in the subject.
41. The composition of aspect 33, wherein a dosage unit contains S-adenosylmethionine (SAM), an Ashwagandha portion or extract thereof, 5-hydroxytryptophane (5-HTP), theanine, gamma-aminobutyric acid (GABA), a Kava portion or extract thereof, a Valerian portion or extract thereof, Mg2+, vitamin B6, vitamin B12, N-acetyltrosine, and/or a St. John's wort portion or extract thereof as the compound or extract and, upon oral administration to a subject, reduces stress in the subject.
42. The composition of aspect 33, wherein a dosage unit contains omega-3 fatty acid, melatonin, a Hops portion or extract thereof, a Chamomile portion or extract thereof, a Valerian portion or extract thereof, a Passion flower portion or extract thereof, and/or a St. John's wort portion or extract thereof as the compound or extract and, upon oral administration to a subject, supports sleep health in the subject.
43. The composition of aspect 33, wherein a dosage unit contains CoQ10, an omega-3 fatty acid, methylsulfonylmethane (MSM), red yeast rice, a phytosterol, a polycosanol, Niacin, a Garlic portion or extract thereof, and/or a Hawthorn portion or extract thereof as the compound or extract and, upon oral administration to a subject, supports heart health in the subject.
44. The composition of aspect 33, wherein a dosage unit contains an alpha lipoic acid, a medium chain fatty acid (MCFA), a short chain fatty acid (SCFA), a conjugated linoleic acid (CLA), a ketone, caffeine, theanine, carnitine, a green tea catechin, ursolic acid, resveratrol, quercetin, and/or a Yohimbe portion or extract thereof as the compound or extract and, upon oral administration to a subject, reduces weight and/or appetite in the subject.
45. The composition of aspect 33, wherein a dosage unit contains Fenugreek portion or extract thereof, a Saw palmetto portion or extract thereof, a Pumpkin portion or extract thereof, a Nettle portion or extract thereof, a Ginger portion or extract thereof, a green tea catechin, lycopene, dehydroepiandrosterone (DHEA), a phytosterol, a Turmeric portion or extract thereof, a Pygeum portion or extract thereof, calcium, zinc, selenium, copper, molybdenum, manganese, boron, Yohimbine, a Ginkgo portion or extract thereof, a Ginseng portion or extract thereof, a Tribulus portion or extract thereof, a Horny goat portion or extract thereof, a Maca portion or extract thereof, a Tongkat Ali portion or extract thereof, vitamin E, vitamin B6, and/or vitamin B12 as the compound or extract and, upon oral administration to a subject, supports men's health in the subject.
46. The composition of aspect 33, wherein a dosage unit contains omega-3 fatty acid, vitamin B1, vitamin B3, vitamin B6, vitamin B7 (biotin), vitamin B9 (folate), vitamin B12, vitamin C, vitamin D3, CoQ10, a probiotic, a Cranberry portion or extract thereof, mannose, a Black cohosh portion or extract thereof, a soy isoflavone, a phytoestrogen, a Wild yam portion or extract thereof, a Chasteberry portion or extract thereof, a Cissus quadrangularis portion or extract thereof, a Licorice portion or extract thereof, a Dong Quai portion or extract thereof, and/or Maca portion or extract thereof as the compound or extract and, upon oral administration to a subject, improves women's health in the subject.
47. The composition of aspect 33, wherein a dosage unit contains omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a Coffee fruit portion or extract thereof, a Ginkgo biloba portion or extract thereof, an alpha lipoic acid, apoaequorin, and/or uridine-5-monophosphateas the compound or extract and, upon oral administration to a subject, supports brain and/or mind health in the subject.
48. The composition of aspect 33, wherein a dosage unit contains dehydroepiandrosterone (DHEA), S-adenosylmethionine (SAM), eicosapentaenoic acid (EPA), a Bacopa monierii portion or extract thereof, caffeine, 5-hydroxytryptophane (5-HTP), tryptophan, and/or a St. John's wort portion or extract thereof as the compound or extract and, upon oral administration to a subject, supports mood in the subject.
49. The composition of aspect 33, wherein a dosage unit contains alpha lipoic acid, a prebiotic, a probiotic, bromelain, papain, a Ginger portion or extract thereof, an amylase, a lipase, a lactase, a galactosidase, a xylanase, a short chain fatty acid (SCFA), N-acetylcysteine, a Milk Thistle portion or extract thereof, a soluble fiber, and/or an insoluble fiber as the compound or extract and, upon oral administration to a subject, improves digestive health in the subject.
50. The composition of aspect 33, wherein a dosage unit contains CoQ10, a ketone, creatine, a betalains, a plant protein, and a whey protein as the compound or extract and, upon oral administration to a subject, improves strength or fitness in the subject.
51. A method of enhancing a biological activity of a nutritionally acceptable compound or extract, comprising: combining the nutritionally acceptable compound or extract with a plurality of chemically distinct polyphenol-containing plant materials or chemically distinct polyphenols from plant materials to thereby form an enhanced composition; wherein the plurality of chemically distinct polyphenol-containing plant materials or chemically distinct polyphenols are present in the enhanced composition in an amount effective to enhance the biological activity of the nutritionally acceptable compound or extract as compared to the biological activity of the nutritionally acceptable compound or extract alone; and wherein the enhanced biological activity is observed in a subject upon oral administration to the subject.
52. The method of aspect 51, wherein the biological activity is support of bone and joint health, and wherein the nutritionally acceptable compound or extract is selected from the group consisting of vitamin D3, vitamin K2, vitamin B3, vitamin A, a green tea catechin, chondroitin, glucosamine, calcium fructoborate, methylsulfonylmethane (MSM), Collagen type II, a hyaluronic acid, L-lysine, an eggshell membrane, calcium, magnesium, zinc, copper, strontium, an omega-3 fatty acid, a turmeric portion or extract thereof, curcumin, a Boswellia portion or extract thereof, and a demineralized bone matrix.
53. The method of aspect 51, wherein the biological activity is support of hair and skin health, and wherein the nutritionally acceptable compound or extract is selected from the group consisting of an alpha lipoic acid, methylsulfonylmethane (MSM), a collagen, keratin, L-cysteine, an Aloe vera portion or extract thereof, a turmeric portion or extract thereof, a ginger portion or extract thereof, an omega-3 fatty acid, a monounsaturated fatty acid (MUFA, and especially oleic acid), a polyunsaturated fatty acid (PUFA), CoQ10, biotin, magnesium, selenium, zinc, copper, iron, silicon, vitamin B3, vitamin B6, vitamin D, vitamin E, biotin, a hyaluronic acid, hydroxyproline, a ceramide, and a Horsetail grass portion or extract thereof.
54. The method of aspect 51, wherein the biological activity is support of cellular energy, and wherein the nutritionally acceptable compound or extract is selected from the group consisting of CoQ10, a medium chain fatty acid (MCT), a ribose, niacin, nicotinamide, nicotinamide riboside, and NAD.
55. The method of aspect 51, wherein the biological activity is reduction in inflammation, and wherein the nutritionally acceptable compound or extract is selected from the group consisting of an omega-3 fatty acid, methylsulfonylmethane (MSM), melatonin, a green tea catechin, ursolic acid, resveratrol, and a lignan.
56. The method of aspect 51, wherein the biological activity is support of immunity, and wherein the nutritionally acceptable compound or extract is selected from the group consisting of an omega-3 fatty acid, Zn+, N-acetylcysteine, a mushroom polysaccharide, an Elderberry portion or extract thereof, Vitamin C, an Echinacea portion or extract thereof, a Garlic portion or extract thereof, a probiotic, colostrum, a beta glucan, a Grapefruit portion or extract thereof, Oscillococcinum, inositol hexaphosphate, and a lignan.
57. The method of aspect 51, wherein the biological activity is reduction in stress, and wherein the nutritionally acceptable compound or extract is selected from the group consisting of S-adenosylmethionine (SAM), an Ashwagandha portion or extract thereof, 5-hydroxytryptophane (5-HTP), theanine, gamma-aminobutyric acid (GABA), a Kava portion or extract thereof, a Valerian portion or extract thereof, Mg2+, vitamin B6, vitamin B12, N-acetyltrosine, and a St. John's wort portion or extract thereof.
58. The method of aspect 51, wherein the biological activity is support of sleep health, and wherein the nutritionally acceptable compound or extract is selected from the group consisting of an omega-3 fatty acid, melatonin, a Hops portion or extract thereof, a Chamomile portion or extract thereof, a Valerian portion or extract thereof, a Passion flower portion or extract thereof, and a St. John's wort portion or extract thereof.
59. The method of aspect 51, wherein the biological activity is support of heart health, and wherein the nutritionally acceptable compound or extract is selected from the group consisting of CoQ10, an omega-3 fatty acid, methylsulfonylmethane (MSM), red yeast rice, a phytosterol, a polycosanol, Niacin, a Garlic portion or extract thereof, and a Hawthorn portion or extract thereof
60. The method of aspect 51, wherein the biological activity is reduction of weight and/or appetite, and wherein the nutritionally acceptable compound or extract is selected from the group consisting of an alpha lipoic acid, a medium chain fatty acid (MCFA), a short chain fatty acid (SCFA), a conjugated linoleic acid (CLA), a ketone, caffeine, theanine, carnitine, a green tea catechin, ursolic acid, resveratrol, quercetin, and a Yohimbe portion or extract thereof.
61. The method of aspect 51, wherein the biological activity is support of men's health, and wherein the nutritionally acceptable compound or extract is selected from the group consisting of a Fenugreek portion or extract thereof, a Saw palmetto portion or extract thereof, a Pumpkin portion or extract thereof, a Nettle portion or extract thereof, a Ginger portion or extract thereof, a green tea catechin, lycopene, dehydroepiandrosterone (DHEA), a phytosterol, a Turmeric portion or extract thereof, a Pygeum portion or extract thereof, calcium, zinc, selenium, copper, molybdenum, manganese, boron, Yohimbine, a Ginkgo portion or extract thereof, a Ginseng portion or extract thereof, a Tribulus portion or extract thereof, a Horny goat portion or extract thereof, a Maca portion or extract thereof, a Tongkat Ali portion or extract thereof, vitamin E, vitamin B6, and vitamin B12.
62. The method of aspect 51, wherein the biological activity is support of woman's health, and wherein the nutritionally acceptable compound or extract is selected from the group consisting of an omega-3 fatty acid, vitamin B1, vitamin B3, vitamin B6, vitamin B7 (biotin), vitamin B9 (folate), vitamin B12, vitamin C, vitamin D3, CoQ10, a probiotic, a Cranberry portion or extract thereof, mannose, a Black cohosh portion or extract thereof, a soy isoflavone, a phytoestrogen, a Wild yam portion or extract thereof, a Chasteberry portion or extract thereof, a Cissus quadrangularis portion or extract thereof, a Licorice portion or extract thereof, a Dong Quai portion or extract thereof, and Maca portion or extract thereof.
63. The method of aspect 51, wherein the biological activity is support of brain and/or mind health, and wherein the nutritionally acceptable compound or extract is selected from the group consisting of an omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a Coffee fruit portion or extract thereof, a Ginkgo biloba portion or extract thereof, an alpha lipoic acid, apoaequorin, and uridine-5-monophosphate.
64. The method of aspect 51, wherein the biological activity is support of mood, and wherein the nutritionally acceptable compound or extract is selected from the group consisting of dehydroepiandrosterone (DHEA), S-adenosylmethionine (SAM), eicosapentaenoic acid (EPA), a Bacopa monierii portion or extract thereof, caffeine, 5-hydroxytryptophane (5-HTP), tryptophan, and a St. John's wort portion or extract thereof
65. The method of aspect 51, wherein the biological activity is support of digestive health, and wherein the nutritionally acceptable compound or extract is selected from the group consisting of an alpha lipoic acid, a prebiotic, a probiotic, bromelain, papain, a Ginger portion or extract thereof, an amylase, a lipase, a lactase, a galactosidase, a xylanase, a short chain fatty acid (SCFA), N-acetylcysteine, a Milk Thistle portion or extract thereof, a soluble fiber, and an insoluble fiber.
66. The method of aspect 51, wherein the biological activity is support of strength and/or fitness, and wherein the nutritionally acceptable compound or extract is selected from the group consisting of CoQ10, a ketone, creatine, a betalains, a plant protein, and a whey protein.
67. The method of any one of aspects 51-66, wherein the plant materials have a red color, a green color, an orange-yellow color, and a purple-blue color.
68. The method of aspect 67, wherein the (a) red colored plant materials comprise an apple extract, a pomegranate extract, a tomato powder, and a beet root powder; (b) green colored plant materials comprise an olive extract, a rosemary extract, a green coffee bean extract, and a kale powder; (c) orange-yellow colored plant materials comprise an onion extract, a ginger extract, a grapefruit extract, and a carrot powder; and (d) purple-blue colored plant materials comprise a grape extract, a blueberry extract, a currant powder, and an elderberry powder.
69. The method of any one of aspects 51-68, wherein the enhanced composition is formulated for oral administration.
70. A method of enhancing a biological activity of a plurality of chemically distinct polyphenol-containing plant materials or chemically distinct polyphenols from plant materials, comprising: combining the plurality of chemically distinct polyphenol-containing plant materials or chemically distinct polyphenols with a nutritionally acceptable compound or extract to thereby form an enhanced composition; wherein the nutritionally acceptable compound or extract is present in the enhanced composition in an amount effective to enhance the biological activity of the plurality of chemically distinct polyphenol-containing plant materials or chemically distinct polyphenols as compared to the plurality of chemically distinct polyphenol-containing plant materials or chemically distinct polyphenols alone; and wherein the enhanced biological activity is observed in a subject upon oral administration to the subject.
71. The method of aspect 70, wherein the biological activity is immune support, metabolic support, support of longevity, support of central nervous system (CNS) function, reduction of an inflammatory response, reduction of effects of cardiovascular disease, and/or reduction of the rate of amyloid beta plaque formation, and wherein the plant materials have a red color, a green color, an orange-yellow color, and a purple-blue color.
72. The method of any one of aspects 70 or 71, wherein the biological activity is an inhibition of ARG-1, ARG-2, SIRT-1, Keap/Nrf-2, BACE-1, Cathepsin S, CDK5, IDO1, IDO2, NAMPT, PCSK9, CD47, CD38, CD39, CD73, JAK1, JAK2, and/or JAK3, and wherein the plant materials have a red color, a green color, an orange-yellow color, and a purple-blue color.
73. The method of any one of aspects 71-72, wherein the (a) red colored plant materials comprise an apple extract, a pomegranate extract, a tomato powder, and a beet root powder; (b) green colored plant materials comprise an olive extract, a rosemary extract, a green coffee bean extract, and a kale powder; (c) orange-yellow colored plant materials comprise an onion extract, a ginger extract, a grapefruit extract, and a carrot powder; and (d) purple-blue colored plant materials comprise a grape extract, a blueberry extract, a currant powder, and an elderberry powder.
74. The method of any one of aspects 71-72, wherein the nutritionally acceptable compound or extract is a vitamin, an isolated or purified compound, an element, a complex organic molecule or biological structure, a plant part and/or extract thereof, and enzyme, and/or a probiotic.
75. The method of aspect 74, wherein the vitamin is selected from the group consisting of vitamin A, vitamin B1, vitamin B3 (niacin) or derivative or metabolite thereof (e.g., nicotinamide, nicotinamide riboside, NAD), vitamin B6, vitamin B7 (biotin), vitamin B9 (folate), vitamin B12, vitamin C, vitamin D3, vitamin E, and vitamin K2, or CoQ10.
76. The method of aspect 74, wherein the isolated or purified compound selected form the group consisting of 5-gamma-aminobutyric acid (GABA), alpha lipoic acid (ALA), apoaequorin, a betalain, caffeine, carnitine, a ceramide, chondroitin, a conjugated linoleic acid (CLA), creatine, curcumin, cysteine, a green tea catechin (EGCG, EC, EGC, GCG, GC), cytidine 5-phosphocholine, dehydroepiandrosterone (DHEA), eicosapentaenoic Acid (EPA), a medium-chain fatty acid (MCFA), a short chain fatty acid (SCFA), an omega-3 fatty acid, a monounsaturated fatty acid (MUFA, and especially oleic acid), a polyunsaturated fatty acid (PUFA), a fructoborate, Glucosamine, hyaluronic acid, hydroxyproline, hydroxytryptophan (5-HTP), a soy isoflavone, inositol hexaphosphate, a ketone (beta hydroxybutyrate, acetoacetate, acetone, . . . ), a lignan (sesamine, etc), lecithin, lycopene, lysine, mannose, melatonin, methylsulfonylmethane (MSM), N-acetyltrosine, theanine, a phytosterol (e.g., beta sitosterol), N-acetyl-carnitine, N-acetylcysteine, N-acetyltyrosine, phosphatidylserine, a phytoestrogen, a polycosanol, quercetin, resveratrol, ribose, S-adenosylmethionine (SAM), Tryptophane, tyrosine, uridine-5-monophosphate, ursolic acid, and yohimbine.
77. The method of aspect 74, wherein the element is in ionic or complexed form and is selected from the group consisting of B, Ca, Cu, Fe, Mb, Mg, Mn, Se, Si, Sr, and Zn.
78. The method of aspect 74, wherein the complex organic molecules or biological structure is selected from the group consisting of collagen, colostrum, a demineralized bone matrix, eggshell membrane, fibers, beta glucans, mushroom polysaccharides, plant or whey protein, prebiotics (e.g., FOS, XOS, HMS) and keratin.
79. The method of aspect 74, wherein the plant part or extract thereof is from a plant selected from the group consisting of Aloe vera, Ashwagandha, Bacopa monierii, Black cohosh, Boswellia, Chamomile, Chasteberry, Cissus quadrangularis, Coffee cherry, Cranberry, Dong Quai, Echinacea, Elderberry, Fenugreek, Garlic, Ginger, Ginkgo biloba, Ginseng, Grapefruit, Hawthorn, Horny goat, Horsetail grass, Hops, Kava, Licorice, Maca, Milk Thistle, Nettle, St. John's wort, Tongkat Ali, Passion flower, Pumpkin, Pygeum, Red yeast rice, Rhodiola, Saw palmetto, Tribulus, Turmeric, Valerian, Wild yam, and Yohimbe.
80. The method of aspect 74, wherein the enzyme is selected from the group consisting of amylase, bromelain, galactosidase, papain, lactase, lipase, and xylanase.
81. The method of aspect 74, wherein the probiotic selected form the group consisting of Lactobacillus acidophilus, Lactobacillus casei, Lactobacillus plantarum, Lactobacillus brevis, Lactobacillus gasseri, Lactobacillus rhamnosus, Bifidobacterium lactis, Bifidobacterium breve, and Bifidobacterium longum.
82. A method of supporting health of an individual, comprising administering to the individual a composition according to any one of aspects 1-50.
83. The method of aspect 82, wherein the composition is administered to thereby provide immune support, metabolic support, support longevity, support central nervous system (CNS) function, reduce an inflammatory response, reduce effects of cardiovascular disease, and reduce the rate of amyloid beta plaque formation.
84. The method of aspect 82, wherein the composition is administered to thereby provide support of bone and joint health, support of hair and skin health, support of cellular energy, reduction in inflammation, support of immunity, reduction in stress, support of sleep health, support of heart health, reduction of weight and/or appetite, support of men's health, support of woman's health, support of brain and/or mind health, support of mood, support of digestive health, and support of strength and/or fitness.
85. The method of aspect 82, wherein the composition is orally administered over at least 30 days and/or at a daily dose of between 50 and 1,000 mg.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical or nutraceutical composition refers to both direct and indirect administration of the pharmaceutical or nutraceutical composition, wherein direct administration of the pharmaceutical or nutraceutical composition is typically performed by a health care professional (e.g., physician, nurse, dietitian, etc.), and wherein indirect administration includes a step of providing or making available the pharmaceutical or nutraceutical composition to the health care professional or individual in need thereof for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. An enhanced nutritional composition, comprising:
a nutritionally acceptable compound or extract having a known or suspected activity selected from the group consisting of support of bone and joint health, support of hair and skin health, support of cellular energy, reduction in inflammation, support of immunity, reduction in stress, support of sleep health, support of heart health, reduction of weight and/or appetite, support of men's health, support of woman's health, support of brain and/or mind health, support of mood, support of digestive health, and support of strength and/or fitness; and
a plurality of chemically distinct polyphenol-containing plant materials or chemically distinct polyphenols from plant materials, wherein the plant materials have a red color, a green color, an orange-yellow color, and a purple-blue color.

2. The enhanced nutritional composition of claim 1, wherein the
(a) red colored plant materials comprise an apple extract, a pomegranate extract, a tomato powder, and/or a beet root powder;
(b) green colored plant materials comprise an olive extract, a rosemary extract, a green coffee bean extract, and/or a kale powder;
(c) orange-yellow colored plant materials comprise an onion extract, a ginger extract, a grapefruit extract, and/or a carrot powder; and
(d) purple-blue colored plant materials comprise a grape extract, a blueberry extract, a currant powder, and/or an elderberry powder.

3. The enhanced nutritional composition of claim 1, wherein the composition is formulated in single dosage units for oral administration.

4. The enhanced nutritional composition of claim 3, wherein the dosage unit contains between 50 and 1,000 mg of the composition.

5. The composition of claim 3, wherein the dosage unit is formulated as a capsule, a gummy, or a powder.

6. The composition of claim 3, wherein a dosage unit contains vitamin D3, vitamin K2, vitamin B3, vitamin A, a green tea catechin, chondroitin, glucosamine, calcium fructoborate, methylsulfonylmethane (MSM), Collagen type II, a hyaluronic acid, L-lysine, an eggshell membrane, calcium, magnesium, zinc, copper, strontium, an omega-3 fatty acid, a turmeric portion or extract thereof, curcumin, a Boswellia portion or extract thereof, and/or a demineralized bone matrix as the compound or extract and, upon oral administration to a subject, supports bone and joint health in the subject.

7. The composition of claim 3, wherein a dosage unit contains alpha lipoic acid, methylsulfonylmethane (MSM), a collagen, keratin, L-cysteine, an Aloe vera portion or extract thereof, a turmeric portion or extract thereof, a ginger portion or extract thereof, an omega-3 fatty acid, a monounsaturated fatty acid (MUFA), a polyunsaturated fatty acid (PUFA), CoQ10, biotin, magnesium, selenium, zinc, copper, iron, silicon, vitamin B3, vitamin B6, vitamin D, vitamin E, biotin, a hyaluronic acid, hydroxyproline, a ceramide, and/or a Horsetail grass portion or extract thereof as the compound or extract and, upon oral administration to a subject, supports hair and skin health in the subject.

8. The composition of claim 3, wherein a dosage unit contains of CoQ10, a medium chain fatty acid (MCT), a ribose, niacin, nicotinamide, nicotinamide riboside, and/or NAD as the compound or extract and, upon oral administration to a subject, supports cellular energy in the subject.

9. The composition of claim 3, wherein a dosage unit contains omega-3 fatty acid, methylsulfonylmethane (MSM), melatonin, a green tea catechin, ursolic acid, resveratrol, and/or a lignan as the compound or extract and, upon oral administration to a subject, reduces inflammation in the subject.

10. The composition of claim 3, wherein a dosage unit contains omega-3 fatty acid, Zn⁺, N-acetylcysteine, a mushroom polysaccharide, an Elderberry portion or extract thereof, Vitamin C, an Echinacea portion or extract thereof, a Garlic portion or extract thereof, a probiotic, colostrum, a beta glucan, a Grapefruit portion or extract thereof, Oscillococcinum, inositol hexaphosphate, and/or a lignan as the compound or extract and, upon oral administration to a subject, supports immunity in the subject.

11. The composition of claim 3, wherein a dosage unit contains S-adenosylmethionine (SAM), an Ashwagandha portion or extract thereof, 5-hydroxytryptophane (5-HTP), theanine, gamma-aminobutyric acid (GABA), a Kava portion or extract thereof, a Valerian portion or extract thereof, Mg′, vitamin B6, vitamin B12, N-acetyltrosine, and/or a St. John's wort portion or extract thereof as the compound or extract and, upon oral administration to a subject, reduces stress in the subject.

12. The composition of claim 3, wherein a dosage unit contains omega-3 fatty acid, melatonin, a Hops portion or extract thereof, a Chamomile portion or extract thereof, a Valerian portion or extract thereof, a Passion flower portion or extract thereof, and/or a St. John's wort portion or extract thereof as the compound or extract and, upon oral administration to a subject, supports sleep health in the subject.

13. The composition of claim 3, wherein a dosage unit contains CoQ10, an omega-3 fatty acid, methylsulfonylmethane (MSM), red yeast rice, a phytosterol, a polycosanol, Niacin, a Garlic portion or extract thereof, and/or a Hawthorn portion or extract thereof as the compound or extract and, upon oral administration to a subject, supports heart health in the subject.

14. The composition of claim 3, wherein a dosage unit contains an alpha lipoic acid, a medium chain fatty acid (MCFA), a short chain fatty acid (SCFA), a conjugated linoleic acid (CLA), a ketone, caffeine, theanine, carnitine, a green tea catechin, ursolic acid, resveratrol, quercetin, and/or a Yohimbe portion or extract thereof as the compound or extract and, upon oral administration to a subject, reduces weight and/or appetite in the subject.

15. The composition of claim 3, wherein a dosage unit contains Fenugreek portion or extract thereof, a Saw palmetto portion or extract thereof, a Pumpkin portion or extract thereof, a Nettle portion or extract thereof, a Ginger portion or extract thereof, a green tea catechin, lycopene, dehydroepiandrosterone (DHEA), a phytosterol, a Turmeric portion or extract thereof, a Pygeum portion or extract thereof, calcium, zinc, selenium, copper, molybdenum, manganese, boron, Yohimbine, a Ginkgo portion or extract thereof, a Ginseng portion or extract thereof, a Tribulus portion or extract thereof, a Horny goat portion or extract thereof, a Maca portion or extract thereof, a Tongkat Ali portion or extract thereof, vitamin E, vitamin B6, and/or vitamin B12 as the compound or extract and, upon oral administration to a subject, supports men's health in the subject.

16. The composition of claim 3, wherein a dosage unit contains omega-3 fatty acid, vitamin B1, vitamin B3, vitamin B6, vitamin B7 (biotin), vitamin B9 (folate), vitamin B12, vitamin C, vitamin D3, CoQ10, a probiotic, a Cranberry portion or extract thereof, mannose, a Black cohosh portion or extract thereof, a soy isoflavone, a phytoestrogen, a Wild yam portion or extract thereof, a Chasteberry portion or extract thereof, a Cissus quadrangularis portion or extract thereof, a Licorice portion or extract thereof, a Dong Quai portion or extract thereof, and/or Maca portion or extract thereof as the compound or extract and, upon oral administration to a subject, improves women's health in the subject.

17. The composition of claim 3, wherein a dosage unit contains omega-3 fatty acid, a lecithin, phosphatidylserine, creatine, theanine, tyrosine, N-acetyl-carnitine, N-acetylcysteine, N-acetyltyrosine, cytidine 5-phosphocholine, caffeine, a Coffee fruit portion or extract thereof, a Ginkgo biloba portion or extract thereof, an alpha lipoic acid, apoaequorin, and/or uridine-5-monophosphateas the compound or extract and, upon oral administration to a subject, supports brain and/or mind health in the subject.

18. The composition of claim 3, wherein a dosage unit contains dehydroepiandrosterone (DHEA), S-adenosylmethionine (SAM), eicosapentaenoic acid (EPA), a Bacopa monierii portion or extract thereof, caffeine, 5-hydroxytryptophane (5-HTP), tryptophan, and/or a St. John's wort portion or extract thereof as the compound or extract and, upon oral administration to a subject, supports mood in the subject.

19. The composition of claim 3, wherein a dosage unit contains alpha lipoic acid, a prebiotic, a probiotic, bromelain, papain, a Ginger portion or extract thereof, an amylase, a lipase, a lactase, a galactosidase, a xylanase, a short chain fatty acid (SCFA), N-acetylcysteine, a Milk Thistle portion or extract thereof, a soluble fiber, and/or an insoluble fiber as the compound or extract and, upon oral administration to a subject, improves digestive health in the subject.

20. The composition of claim 3, wherein a dosage unit contains CoQ10, a ketone, creatine, a betalains, a plant protein, and a whey protein as the compound or extract and, upon oral administration to a subject, improves strength or fitness in the subject.
